(19) United States
(12) Patent Application Publication
(10) Pub. No.: US 2024/0035090 A1
(43) Pub. Date: Feb. 1, 2024

(54) mRNA BIOMARKERS FOR DIAGNOSIS OF LIVER DISEASE

(71) Applicant: HEPGENE, INC., Fairfield, CT (US)

(72) Inventors: Patrick Lilley, Fairfield, CT (US); Martin J. Keiser, III, Fairfield, CT (US)

(73) Assignee: HEPGENE, INC., Fairfield, CT (US)

(21) Appl. No.: 18/176,124

(22) Filed: Feb. 28, 2023

Related U.S. Application Data
(63) Continuation of application No. PCT/US2021/047760, filed on Aug. 26, 2021.
(60) Provisional application No. 63/072,071, filed on Aug. 28, 2020.

(51) Int. Cl. C12Q 1/6883; G16B 25/10; G16H 50/20

(52) U.S. Cl. CPC C12Q 1/6883; G16B 25/10; G16H 50/20; C12Q 2600/118; C12Q 2600/158; C12Q 2600/178

(57) ABSTRACT

Embodiments include a system and method of using biomarkers in the diagnosis of liver disease. A subject can be screened based on expression of specific mRNAs, miRNAs, proteins or peptides in blood, serum or plasma. Specific mRNAs/miRNAs are used as biomarkers to distinguish healthy individuals from individuals affected with a liver disease. Embodiments include 14 mRNA biomarkers to diagnose NAFL vs. healthy liver (i.e. early detection of liver disease). Embodiments also include 9 mRNA biomarkers to diagnose NAFL vs. NASH (i.e. stage of liver disease progression) and 37 mRNA biomarkers to diagnose NASH. Further embodiments include 32 miRNA biomarkers to diagnose and distinguish between NASH, hepatitis B and hepatitis C. Levels of more than one of the mRNAs, miRNAs or proteins can be scored and compared to one or more threshold values to diagnose or determine the prognosis of a liver disease. Embodiments also include a kit for screening healthy subjects from subjects affected with a liver disease.

### PRIORITY CLAIM

This application is a continuation of International Patent Application No. PCT/US2021/047760, filed Aug. 26, 2021, which claims the benefit of U.S. Provisional Application No. 63/072,071, filed Aug. 28, 2020, both of which are incorporated herein by reference in their entirety, including drawings.

### FIELD OF THE INVENTION

The invention relates to the diagnosis of disease using biomarkers, and more specifically, to a system and method of diagnosing liver diseases based on specific mRNAs and/or miRNAs with altered expression levels.

### BACKGROUND

In humans, the liver performs several vital functions. It detoxifies various metabolites, synthesizes proteins and produces biochemicals necessary for digestion and growth. Liver damage can be fatal as there is no means to compensate for the absence of liver function. Liver dialysis can be used in the short term as a bridge to transplantation or liver regeneration. However, it cannot support a patient for an extended period of time. Liver transplantation, a complex and risky procedures, is the only option for liver failure.

Liver disease (also called hepatic disease) is a type of damage to or disease of the liver. Chronic liver disease ensues when the disease is ongoing. There are several known causes of liver damage including infections (e.g. hepatitis), autoimmune diseases, cancer, alcohol/drug use, and inherited disorders (e.g. hemochromatosis). Liver disease is often categorized into four stages. The first stage is inflammation in which the liver is enlarged or inflamed. The next stage is fibrosis when scar tissue begins to replace healthy tissue in the inflamed liver. The third stage is cirrhosis when scarring becomes severe, making it difficult for the liver to function properly. In end-stage liver disease (ESLD), liver function has deteriorated to the point where the damage is irreversible. The only option for a patient with ESLD is a liver transplant.

Fatty liver disease (FLD) is a condition in which excess fat builds up in the liver. FLD can lead to the most severe form of the disease referred to as nonalcoholic steatohepatitis (NASH). Complications of FLD include cirrhosis, liver cancer and esophageal varices. There are two types of FLD: non-alcoholic fatty liver disease (NAFLD) and alcoholic liver disease.

Simple fatty liver (NAFL) is defined as hepatic steatosis with no evidence of hepatocellular injury. Both NAFL and NASH can be defined as the liver manifestation of a metabolic disorder. NASH is closely related to the triple epidemic of obesity, pre-diabetes, and diabetes. Its symptoms are often silent or non-specific, making it difficult to diagnose. As a result, patients with NASH are often unaware of their condition until late stages of the disease when mitigation and treatment options are limited. Thus, liver testing is important for patients at risk of developing NAFL and NASH.

Liver function tests (LFTs) are often used to test the proper function of the liver. LFTs check levels of proteins such as alanine transaminase (ALT), aspartate aminotransferase (AST), alkaline phosphatase (ALP), albumin and bilirubin. High levels of ALT, AST or ALP can be a sign of liver damage. Similarly, a high result on the bilirubin test can indicate poor liver function. An albumin test measures how well a patient's liver is making this particular protein. A low result on this test can indicate poor liver function. Other common liver tests are visual and often subjective.

Imaging tests such as transient elastography, ultrasound and magnetic resonance imaging (MRI) can be used to visually examine the liver tissue and the bile ducts. Abdominal Ultrasound uses sound waves to produce images to evaluate the size and shape of the liver, as well as blood flow through the liver. A liver biopsy is often performed after persistent abnormal liver blood tests (liver enzymes), unexplained yellowing of the skin (jaundice), a liver abnormality found on ultrasound, CT scan, or nuclear scan and/or unexplained enlargement of the liver. A liver biopsy entails inserting a needle into the liver to collect a tissue sample. A liver biopsy can also be used to estimate the degree of liver damage, to grade and stage hepatitis B and C, and to determine the best treatment for the damage or disease.

These conventional liver diagnostic tools have limitations. Recent studies show that NAFL (i.e. the early stages of the disease) is not accurately detected by routine liver enzyme tests. The asymptomatic nature of the disease and fact that it is difficult to detect results in a higher likelihood that a patient progresses in severity to NASH. Diagnosis of steatohepatitis is difficult and typically requires a liver biopsy (for example, NASH refers to findings on liver biopsy in patients with steatohepatitis in the absence of significant alcohol consumption). Therefore, liver biopsy remains the only means of assessing the presence and extent of specific necroinflammatory changes and fibrosis in steatohepatitis.

Liver biopsy is invasive, expensive and highly variable. Diagnostic variability from the same pathologist analyzing tissue collected from the same patient can be as high as 20% and as high as 50% between two pathologists for the same patient. Studies demonstrate the negative predictive value for the tissue biopsy in the 70% range. Alternative approaches use surrogate markers, such as aminotransferases and fibrosis markers. However, these markers are not adequate for monitoring diseases as is necessary for clinical studies.

Because of these limitations, drug developers often focus on treating certain aspects of the disease and targeting conditions like fibrosis as a surrogate endpoint in their trials. As a result, most blood-based tests available for clinical research are designed to diagnose the various levels of severity of fibrosis. However, several failures have recently called this modality into question by regulatory agencies. Further, experts have questioned whether a reduction in fibrosis effectively improves the underlying NASH condition. Those involved in clinical trials have faced the notion that fibrosis may not be an appropriate surrogate endpoint.

Due to its high prevalence and potential for severe hepatic outcomes such as liver cirrhosis, fatty liver disease has become a major issue for the society and health care. Up to 30% of the general population is affected by non-alcoholic fatty liver disease (NAFLD), reaching up to 70% among diabetic patients. Because of the inability to accurately diagnose NAFL/NASH, it is difficult to study disease progression and develop drugs/therapies. Moreover, it is difficult for drug developers to properly recruit patients for their clinical trials. Further, patients are generally averse to undergoing liver biopsy because of its invasiveness and risks. Accordingly, there is a desperate need for accurate and affordable noninvasive liver tests.

Recent studies have provided evidence of abnormal expression patterns of mRNAs and miRNAs in patients with liver disease. This presents the possibility of their use as diagnostic and prognostic biomarkers. However, diagnostic assays have been ineffective or unreliable in part because they rely on a single molecular marker. Because a single marker is used, such methods have not enabled reliable predictions of the presence of liver disease. Thus, there is a need for the identification of multiple molecular biomarkers that overcome these limitations by accurately and reliably diagnosing liver disease.

### SUMMARY OF THE INVENTION

The following summary is provided to facilitate an understanding of some of the innovative features unique to the disclosed embodiment and is not intended to be a full description. A full appreciation of the various aspects of the embodiments disclosed herein can be gained by taking into consideration the entire specification, claims and abstract as a whole.

The invention relates to a method for diagnosing a liver disease, or a predisposition to a liver disease in a patient comprising steps of (a) determining in a sample of a patient suspected to suffer from a liver disease the amount of at least one biomarker from Tables 1, 2, 3 or 4 and (b) comparing the amount of the at least one biomarker with a reference, whereby a liver disease or a predisposition is to be diagnosed.

The methods described herein can use one or more of the biomarkers that include, but are not limited to, mRNA probes set forth in Tables 1-3 and miRNAs set forth in Table 4.

Additional embodiments include a system and method of detection and diagnosis of early stages of liver disease. Embodiments also include a system and method of detection and diagnosis of liver disease that is not identifiable by conventional methods (e.g. LFT's, imaging and biopsy). Embodiments further include a system and method of detecting/monitoring the progression of liver disease, including stages of the disease. The systems and methods can utilize biomarkers along with additional biomedical information of a patient.

Embodiments include a system and method of detection and diagnosis of nonalcoholic fatty liver (“NAFL”) and nonalcoholic steatohepatitis (“NASH”) (i.e. liver disease progression).

Embodiments include mRNA biomarkers to diagnose non-alcoholic fatty liver (NAFL). Embodiments also include mRNA biomarkers to diagnose early stages of non-alcoholic fatty liver (NAFL).

Embodiments include mRNA biomarkers to diagnose liver disease and distinguish between NAFL and non-alcoholic steatohepatitis (NASH). Additional embodiments include mRNA biomarkers to distinguish between stages of liver disease.

Embodiments include mRNA biomarkers to diagnose NASH vs. healthy liver.

Embodiments also include miRNA biomarkers to distinguish a health liver from a liver with NASH, hepatitis B or hepatitis (i.e. using binary classifiers and four-state classifiers).

Embodiments include mRNA biomarkers to monitor the progress of liver disease in a patient. Embodiments include mRNA biomarkers to provide guidance in choosing among one or more therapies and/or drugs to treat a patient with liver disease.

Embodiments include a system and method of determining a preferred treatment for a patient suffering from liver disease. Embodiments also include a system and method of determining a patient's likelihood of responding favorably to surgical procedures such as bariatric surgery.

Embodiments include a method that uses one or more algorithms to diagnose a liver disease based on levels of one or more mRNA biomarkers.

Embodiments include a method that uses one or more algorithms to diagnose a liver disease based on levels of one or more miRNA biomarkers.

Embodiments include a method that uses one or more algorithms to diagnose a liver disease based on levels of one or more protein biomarkers.

Embodiments include a method of diagnosing a liver disease or determining a prognosis of a subject with a liver disease that includes steps of (a) measuring expression levels of at least two miRNAs, mRNAs, proteins or peptides in a test sample from the subject, (b) receiving the expression levels with a computer, (c) compiling the expression levels to yield a score, and (d) comparing the score to one or more threshold values to diagnose or determine the prognosis of liver disease.

Embodiments include a method of diagnosing a liver disease or determining a prognosis of a patient with a liver disease that includes steps of: (a) measuring expression levels of at least two miRNAs, mRNAs, proteins or peptides in samples from subjects with a liver disease, (b) measuring expression levels of the same at least two miRNAs, mRNAs, proteins or peptides in samples from healthy patients, (c) calculating one or more threshold values based on the expression levels of a) and the expression levels of b), (d) creating a score from the measured levels of the at least two miRNAs, mRNAs, proteins or peptides in samples from a test patient, and (e) diagnosing or determining the prognosis of a liver disease in the test patient by comparing the score to the one or more threshold values.

Embodiments include a method of diagnosing liver disease or determining a prognosis of a subject with liver disease (such as NAFL), comprising steps of a) measuring the expression level of at least one mRNA in a test sample from plasma of the subject; b) comparing the expression level of the at least one mRNA in the test sample to a level in a base sample; and c) diagnosing or determining the prognosis of liver disease based on altered expression the mRNA in the test sample.

Embodiments include a method of diagnosing liver disease or determining a prognosis of a subject with liver disease (such as NASH), comprising steps of a) measuring the expression level of at least one miRNA in a test sample from plasma of the subject; b) comparing the expression level of the at least one miRNA in the test sample to a level in a base sample; and c) diagnosing or determining the prognosis of liver disease based on altered expression the miRNA in the test sample.

Embodiments further include a method of diagnosing and characterizing a liver disease (e.g. distinguishing NASH v. hepatitis B v. hepatitis C and healthy liver), comprising steps of a) measuring the expression level of at least one miRNA in a test sample from plasma of the subject; b) comparing the expression level of the at least one miRNA in the test sample to a level in a base sample; and c) diagnosing or determining the prognosis of liver disease based on altered expression the miRNA in the test sample.

Embodiments include a method of diagnosing liver disease or determining a prognosis of a subject with liver disease (such as NASH), comprising steps of a) measuring the expression level of at least one protein or peptide fragment in a test sample from plasma of the subject; b) comparing the expression level of the at least one protein or peptide fragment in the test sample to a level in a base sample; and c) diagnosing or determining the prognosis of liver disease based on altered expression the protein or peptide fragment in the test sample.

Embodiments also include a method of diagnosing liver disease or determining a prognosis of a test subject with liver disease (such as NAFL), comprising steps of: a) measuring expression levels of two or more mRNAs in plasma samples from subjects with liver disease; b) measuring expression levels of the two or more mRNAs in plasma samples from healthy subjects; c) comparing the expression levels of the two or more mRNAs in the plasma samples from the subjects with liver disease to the levels in the plasma samples from the healthy subjects; d) identifying mRNAs that have altered levels of expression in the plasma samples from the subjects with liver disease; e) creating a biomarker fingerprint from the mRNAs with altered levels of expression; and f) diagnosing or determining the prognosis of liver disease in the test subject by comparing of levels of mRNAs from plasma of the test subject to those in the biomarker fingerprint.

Embodiments also include a method of diagnosing liver disease or determining a prognosis of a test subject with liver disease (such as NAFL), comprising steps of: a) measuring expression levels of two or more mRNAs in buffy coat obtained from blood from subjects with liver disease; b) measuring expression levels of the two or more mRNAs in buffy coat obtained from blood from samples from healthy subjects; c) comparing the expression levels of the two or more mRNAs in the buffy coat obtained from blood from samples from the subjects with liver disease to the levels in the plasma samples from the healthy subjects; d) identifying mRNAs that have altered levels of expression in the buffy coat obtained from blood from samples from the subjects with liver disease; e) creating a biomarker fingerprint from the mRNAs with altered levels of expression; and f) diagnosing or determining the prognosis of liver disease in the test subject by comparing of levels of mRNAs from plasma of the test subject to those in the biomarker fingerprint.

Embodiments also include a method of diagnosing liver disease or determining a prognosis of a test subject with liver disease (such as NASH, hepatitis B or hepatitis C), comprising steps of: a) measuring expression levels of two or more miRNAs in buffy coat obtained from blood from subjects with liver disease; b) measuring expression levels of the two or more miRNAs in buffy coat obtained from blood from samples from healthy subjects; c) comparing the expression levels of the two or more miRNAs in the buffy coat obtained from blood from samples from the subjects with liver disease to the levels in the plasma samples from the healthy subjects; d) identifying miRNAs that have altered levels of expression in the buffy coat obtained from blood from samples from the subjects with liver disease; e) creating a biomarker fingerprint from the miRNAs with altered levels of expression; and f) diagnosing or determining the prognosis of liver disease in the test subject by comparing of levels of miRNAs from plasma of the test subject to those in the bio marker fingerprint.

Embodiments also include a method of diagnosing liver disease or determining a prognosis of a test subject with liver disease (such as NAFL), comprising steps of: a) measuring expression levels of two or more mRNAs in serum obtained from blood from subjects with liver disease; b) measuring expression levels of the two or more mRNAs in serum obtained from blood from samples from healthy subjects; c) comparing the expression levels of the two or more mRNAs in the serum obtained from blood from samples from the subjects with liver disease to the levels in the plasma samples from the healthy subjects; d) identifying mRNAs that have altered levels of expression in the serum obtained from blood from samples from the subjects with liver disease; e) creating a biomarker fingerprint from the mRNAs with altered levels of expression; and f) diagnosing or determining the prognosis of liver disease in the test subject by comparing of levels of mRNAs from plasma of the test subject to those in the biomarker fingerprint.

Embodiments also include a method of diagnosing liver disease or determining a prognosis of a test subject with liver disease (such as NASH, hepatitis B or hepatitis C), comprising steps of: a) measuring expression levels of two or more miRNAs in serum obtained from blood from subjects with liver disease; b) measuring expression levels of the two or more miRNAs in serum obtained from blood from samples from healthy subjects; c) comparing the expression levels of the two or more miRNAs in the serum obtained from blood from samples from the subjects with liver disease to the levels in the plasma samples from the healthy subjects; d) identifying miRNAs that have altered levels of expression in the serum obtained from blood from samples from the subjects with liver disease; e) creating a biomarker fingerprint from the miRNAs with altered levels of expression; and f) diagnosing or determining the prognosis of liver disease in the test subject by comparing of levels of miRNAs from plasma of the test subject to those in the biomarker fingerprint.

Embodiments also include a diagnostic kit for diagnosing liver disease, wherein the kit comprises a plurality of nucleic acid molecules, each nucleic acid molecule encoding a mRNA sequence. The nucleic acid molecules identify variations in expression levels of one or more mRNAs in a plasma sample from a test subject. The expression levels of one or more mRNAs can indicate healthy liver function or the presence of a liver disease

Embodiments also include a diagnostic kit for diagnosing liver disease, wherein the kit comprises a plurality of nucleic acid molecules, each nucleic acid molecule encoding a miRNA sequence. The nucleic acid molecules identify variations in expression levels of one or more miRNAs in a plasma sample from a test subject. The expression levels of one or more miRNAs can indicate healthy liver function or the presence of a liver disease

### BRIEF DESCRIPTION OF THE FIGURES

FIG. 1 depicts a method of combining results from biomarkers to achieve a final categorical determination.

### DEFINITIONS

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can be in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

All numerical designations, e.g., pH, temperature, time, concentration, and molecular weight, including ranges, are to be understood as approximations in accordance with common practice in the art. When used herein, the term “about” may connote variation (+) or (−) 1%, 5% or 10% of the stated amount, as appropriate given the context. It is to be understood, although not always explicitly stated, that the reagents described herein are merely exemplary and that equivalents of such are known in the art.

The term “algorithm” refers to a specific set of instructions or a definite list of well-defined instructions for carrying out a procedure, typically proceeding through a well-defined series of successive states, and eventually terminating in an end-state.

The term “biomarker” refers generally to a DNA, RNA, protein, carbohydrate, or glycolipid-based molecular marker, the expression or presence of which in a subject's sample can be detected by standard methods (or methods disclosed herein) and is predictive or prognostic of the effective responsiveness or sensitivity of a mammalians subject with liver disease. Biomarkers may be present in a test sample but absent in a control sample, absent in a test sample but present in a control sample, or the amount or of biomarker can differ between a test sample and a control sample. For example, genetic biomarkers assessed (e.g., specific mutations and/or SNPs) can be present in such a sample, but not in a control sample, or certain biomarkers are seropositive in the sample, but seronegative in a control sample. Also, optionally, expression of such a biomarker may be determined to be higher than that observed for a control sample. The terms “marker” and “biomarker” are used herein interchangeably.

As used herein, “additional biomedical information” refers to one or more evaluations of an individual, other than using any of the biomarkers described herein, that are associated with liver disease or the risk of liver disease. “Additional biomedical information” includes any of the following: physical descriptors of an individual, the height and/or weight of an individual (including obesity), the gender of an individual, the ethnicity of an individual, history of drug/alcohol use, occupational history, exposure to known liver toxins (e.g., exposure to any of carbon tetrachloride, vinyl chloride, the herbicide paraquat and/or industrial chemicals called polychlorinated biphenyls), family history of liver disease and the like. Long term use of over-the-counter pain relievers can also cause liver damage. For example, non-prescription pain relievers such as acetaminophen (Tylenol, others), aspirin, ibuprofen (Advil, Motrin IB, others) and naproxen (Aleve, others) can damage your liver, especially if taken frequently and/or combined with alcohol. Prescription medications can also cause liver damage. Some medications linked to liver injury include the statin drugs used to treat high cholesterol, the combination drug amoxicillin-clavulanate (Augmentin), phenytoin (Dilantin, Phenytek), azathioprine (Azasan, Imuran), niacin (Niaspan), ketoconazole, certain antivirals and anabolic steroids and the like. Long term use of certain herbs and supplements is also associated with liver damage. For example, aloe vera, black cohosh, cascara, chaparral, comfrey, kava and ephedra and the like. Additional biomedical information can also be obtained from routine imaging techniques, including CT imaging (e.g., low-dose CT imaging) and X-ray. Testing of biomarker levels in combination with an evaluation of any additional biomedical information can, for example, improve sensitivity, specificity, and/or AUC for detecting liver disease (or the identifying a patient who is at risk of liver disease) as compared to biomarker testing alone or evaluating any particular item of additional biomedical information alone (e.g., CT imaging alone).

The term “area under the curve” or “AUC” refers to the area under the curve of a receiver operating characteristic (ROC) curve, both of which are well known in the art. AUC measures are useful for comparing the accuracy of a classifier across the complete data range. Classifiers with a greater AUC have a greater capacity to classify unknowns correctly between two groups of interest (e.g., liver disease samples and normal or control samples). ROC curves are useful for plotting the performance of a particular feature (e.g., any of the biomarkers described herein and/or any item of additional biomedical information) in distinguishing between two populations (e.g., cases having liver disease and controls without liver disease). Typically, the feature data across the entire population (e.g., the cases and controls) are sorted in ascending order based on the value of a single feature. Then, for each value for that feature, the true positive and false positive rates for the data are calculated. The true positive rate is determined by counting the number of cases above the value for that feature and then dividing by the total number of cases. The false positive rate is determined by counting the number of controls above the value for that feature and then dividing by the total number of controls. Although this definition refers to scenarios in which a feature is elevated in cases compared to controls, this definition also applies to scenarios in which a feature is lower in cases compared to the controls (in such a scenario, samples below the value for that feature would be counted). ROC curves can be generated for a single feature as well as for other single outputs, for example, a combination of two or more features can be mathematically combined (e.g., added, subtracted, multiplied, etc.) to provide a single sum value, and this single sum value can be plotted in a ROC curve. Additionally, any combination of multiple features, in which the combination derives a single output value, can be plotted in a ROC curve. These combinations of features may comprise a test. The ROC curve is the plot of the true positive rate (sensitivity) of a test against the false positive rate (1-specificity) of the test.

The term “binary classification” refers to the task of classifying the elements of a given set into two groups (predicting which group each one belongs to) on the basis of a classification rule. A decision as to whether or not an item has some specified characteristic (some typical binary classification) includes medical testing to determine if a patient has particular disease (i.e. liver disease) or not—the classification property is the presence of the disease. Similarly, “four-state classification” refers to the task of classifying elements of a given set into four groups on the basis of a classification rule. It can be used as described herein to categorize a patient as one of (1) healthy liver, (2) NASH, (3) hepatitis B or (4) hepatitis C.

As used herein, “detecting” or “determining” with respect to a biomarker value includes the use of both the instrument required to observe and record a signal corresponding to a biomarker value and the material/s required to generate that signal. In various embodiments, the biomarker value is detected using any suitable method, including fluorescence, chemiluminescence, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like.

The term “fingerprint,” “disease fingerprint,” or “biomarker signature” refers to a plurality or pattern of biomarkers that have elevated or reduced levels in a subject with disease. A fingerprint can be generated by comparing subjects with the disease to healthy subjects and used for screening/diagnosis of the disease.

The term “messenger RNA” or “mRNA” refers to a single-stranded molecule of RNA that corresponds to the genetic sequence of a gene and is read by a ribosome in the process of synthesizing a protein. As used herein, mRNA can also include “miRNA” and small interfering RNAs (siRNAs).

The term “miRNA” or “micro RNA,” “miRNA biomarkers,” or “MicroRNAs” refers to small non-coding RNA molecules (containing about 22 nucleotides) found in plants, animals and some viruses, that function in RNA silencing and post-transcriptional regulation of gene expression. miRNAs function via base-pairing with complementary sequences within mRNA molecules. As a result, these mRNA molecules are silenced, by one or more of the following processes: (1) Cleavage of the mRNA strand into two pieces, (2) Destabilization of the mRNA through shortening of its poly(A) tail, and (3) Less efficient translation of the mRNA into proteins by ribosomes. miRNAs can be used as serum diagnostic biomarkers for diseases including liver disease.

An mRNA that is “unregulated” generally refers to an increase in the level of express of the mRNA in response to a given treatment or condition. An mRNA that is “downregulated” generally refers to a “decrease” in the level of expression of the mRNA in response to a given treatment or condition. In some situations, the mRNA level can remain unchanged upon a given treatment or condition. An mRNA from a patient sample can be “unregulated,” i.e., the level of mRNA can be increased, for example, by about 5%, about 10%, about 20%, about 30%, about 40%, about 50%, about 60%, about 70%, about 90%, about 100%, about 200%, about 300%, about 500%, about 1,000%, about 5,000% or more of the comparative control mRNA level or a reference level. Alternatively, an mRNA can be “downregulated,” i.e., the level of mRNA level can be decreased, for example, by about 99%, about 95%, about 90%, about 80%, about 70%, about 60%, about 50%, about 40%, about 30%, about 20%, about 10%, about 5%, about 2%, about 1% or less of the comparative control mRNA level or a reference level.

Similarly, the level of a polypeptide, protein, or peptide from a patient sample can be increased as compared to a control or a reference level. This increase can be about 5%, about 10%, about 20%, about 30%, about 40%, about 50%, about 60%, about 70%, about 90%, about 100%, about 200%, about 300%, about 500%, about 1,000%, about 5,000% or more of the comparative control protein level or a reference level. Alternatively, the level of a protein biomarker can be decreased. This decrease can be, for example, present at a level of about 99%, about 95%, about 90%, about 80%, about 70%, about 60%, about 50%, about 40%, about 30%, about 20%, about 10%, about 5%, about 2%, about 1% or less of the comparative control protein level or a reference level.

The term “nucleic acid probe” or “oligonucleotide probe” refers to a nucleic acid capable of binding to a target nucleic acid of complementary sequence, such as the mRNA biomarkers provided herein, through one or more types of chemical bonds, usually through complementary base pairing, usually through hydrogen bond formation. As used herein, a probe may include natural (e.g., A, G, C, or T) or modified bases (7-deazaguanosine, inosine, etc.). In addition, the bases in a probe may be joined by a linkage other than a phosphodiester bond, so long as it does not interfere with hybridization. It will be understood by one of skill in the art that probes may bind target sequences lacking complete complementarity with the probe sequence depending upon the stringency of the hybridization conditions. The probes are preferably directly labeled with isotopes, for example, chromophores, lumiphores, chromogens, or indirectly labeled with biotin to which a streptavidin complex may later bind. By assaying for the presence or absence of the probe, one can detect the presence or absence of a target mRNA biomarker of interest.

The term “probe set identifier,” or “Affymetrix probe set ID” refers to the identifier that refers to a set of probe pairs selected to represent expressed sequences on an array. (_at =all the probes hit one known transcript; _a=all probes in the set hit alternate transcripts from the same gene; _s=all probes in the set hit transcripts from different genes; _x=some probes hit transcripts from different genes).

The term “steatosis” refers to an abnormal retention of fat (i.e. lipids) within a cell or organ. Steatosis most often affects the liver and is referred to as fatty liver disease.

The term “fatty liver disease,” “FLD,” or hepatic steatosis refers to a condition where excess fat builds up in the liver. FLD often has no or few symptoms. Symptoms can include tiredness or pain in the upper right side of the abdomen. FLD can lead to the most severe form of the disease referred to as NASH. Complications of FLD include cirrhosis, liver cancer and esophageal varices. There are two types of FLD: non-alcoholic fatty liver disease (NAFLD) and alcoholic liver disease.

The term “nonalcoholic fatty liver disease” or “NAFLD” refers to a build-up of fat within the liver cells. It is usually seen in people who are overweight or obese. The early stages of the disease is known as simple fatty liver or steatosis. The fat deposits can continue to build up and eventually lead to scarring of the liver and loss of liver function.

The term “simple fatty liver,” “nonalcoholic fatty liver” or “NAFL” refers to a form of NAFLD in where there are fat deposits in the liver but little or no inflammation or liver cell damage. It can be considered the earliest stage of NAFLD.

The term “non-alcoholic steatohepatitis” or “NASH” refers to a condition in which fat builds up in the liver and eventually causes scar tissue. NASH appears to be associated with obesity (40% of NASH patients), diabetes, protein malnutrition, coronary artery disease, and treatment with steroid medications. It is similar to alcoholic liver disease in patients with no history of alcoholism.

The term “cirrhosis,” “liver cirrhosis” or “hepatic cirrhosis” refers to a condition in which the liver does not function properly due to long-term damage. This damage is characterized by the replacement of normal liver tissue by scar tissue (i.e. fibrosis). The disease generally develops slowly over months or years, often with no symptoms. Eventually, excessive scar formation will result in loss of liver function.

The term “plasma” or “blood plasma” refers to the liquid portion of the blood that carries cells and proteins throughout the body. Plasma can be separated from the blood by spinning a tube of fresh blood containing an anticoagulant in a centrifuge until the blood cells fall to the bottom of the tube.

The term “PCR” or “polymerase chain reaction” refers to a common method used to make many copies of a specific DNA segment. Variations of the technique can be used to determine the presence and amount of one or more miRNAs in a sample. For example, a hydrolysis probe-based stem—loop quantitative reverse-transcription PCR (RT-qPCR) assay can be conducted to confirm and/or quantify the concentrations of selected miRNAs in serum samples from patients and controls.

The term “sample” refers to a biological sample obtained from an individual, body fluid, body tissue, cell line, tissue culture, or other source. Body fluids are, for example, lymph, sera, whole fresh blood, peripheral blood mononuclear cells, frozen whole blood, plasma (including fresh or frozen), urine, saliva, semen, synovial fluid and spinal fluid. Samples also include synovial tissue, skin, hair follicle, and bone marrow. Methods for obtaining tissue biopsies and body fluids from mammals are well known in the art.

The term “subject” or “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human.

The term “prognosis” refers to the forecast or likely outcome of a disease. As used herein, it refers to the probable outcome of liver disease, including whether the disease (e.g. NAFL or NASH) will respond to treatment or mitigation efforts and/or the likelihood that the disease will progress.

“Optional” or “optionally” as used herein means that the subsequently described circumstance may or may not occur, so that the description includes instances where the circumstance occurs and instances where it does not.

Other technical terms used herein have their ordinary meaning in the art that they are used, as exemplified by a variety of technical dictionaries. The particular values and configurations discussed in these non-limiting examples can be varied and are cited merely to illustrate at least one embodiment and are not intended to limit the scope thereof.

### DETAILED DESCRIPTION

It is to be understood that both the foregoing general description and the following detailed description are exemplary and explanatory and are intended to provide further explanation of the subject technology as claimed. Additional features and advantages of the subject technology are set forth in the description below, and in part will be apparent from the description, or may be learned by practice of the subject technology. The advantages of the subject technology will be realized and attained by the structure particularly pointed out in the written description and claims hereof.

Conventional methods of diagnosing early stages of liver disease are generally unreliable. Common liver function tests (LFTs) and imaging tests generally diagnose diseases at advance stages. Further, LFTs measure impaired liver function without a means of indicating a cause or particular liver disease. Liver biopsies are prone to error as they rely on subjective observations by healthcare providers. Other methods rely on the observation of a particular endpoint such as fibrosis. Such endpoints are reached in advanced stages of liver disease when treatments may be ineffective.

The inventors have discovered differences in mRNA expression between healthy patients and those with liver disease. Specific mRNAs are aberrantly expressed in diseased liver as compared to healthy livers. Specific miRNAs can also be expressed at different levels in diseased and healthy livers. The mRNAs/miRNAs can be detected in the plasma of patients. Thus, the present invention is based in part on the finding that liver disease can be reliably identified and different subtypes of liver disease can be distinguished based on particular mRNA/miRNA expression profiles with high sensitivity and specificity. The expression of biomarkers typically includes both up- and down-regulated levels of mRNAs/miRNAs. An analysis of mRNA/miRNA expression biomarkers allows for creation of a “fingerprint” by analyzing mRNA/miRNA expression patterns in diseased and healthy subjects. Thereafter, individual mRNA/miRNA expression levels can be used for the detection of liver disease in different disease stages. The differences can be detected at early stages of liver disease. The biomarkers can also be used to distinguish different subtypes of liver disease from one another and monitor the progress of a liver disease. In another embodiment, the proteins transcribed from identified mRNAs are used as biomarkers.

Early diagnosis of liver disease allows intervention and/or treatment to avoid further liver damage. For example, the biomarkers described herein can be measured and analyzed to determine whether a patient has a healthy liver or is in early stages of liver disease (e.g. NAFL). Conventional methods of diagnosing liver disease rely on comparing LFT's and/or observing fibrotic tissue. Because NAFL is often asymptomatic, these methods may be ineffective in patients with early stages of liver disease. Early, accurate and reliable detection of liver disease can be utilized by researchers and drug developers to recruit patients for clinical trials, support their drug through development, and support the drug post-approval.

The disclosed biomarkers can also be used to monitor the progression of liver disease. For example, the biomarkers described herein can be measured and analyzed to identify the stage of liver disease. Fatty liver (“NAFL”) often progresses to nonalcoholic steatohepatitis (“NASH”). Both NAFL and NASH can be diagnosed and distinguished from one another. When possible, efforts can be made to slow (or reverse) the progress of the disease. Conventional methods lack the reliability and sensitivity to distinguish between these conditions and monitor progression.

Embodiments include a set of diagnostic markers or a molecular fingerprint, for reliable identification and/or treatment of patients with liver disease. Embodiments further include methods of diagnosing liver disease based on specific mRNAs/miRNAs that have altered expression levels. While individual mRNAs can be monitored, the invention includes multiple mRNAs/miRNAs of particular value as biomarkers to screen or distinguish healthy individuals from individuals affected with disease.

The disclosed biomarkers can also be used to distinguish liver diseases from one another. For example, the biomarkers described herein can be measured and analyzed to distinguish a healthy liver from one with NASH, hepatitis B and hepatitis C (i.e. binary classifiers and a four-state classifier). The biomarkers can also indicate the extent of liver damage. This can help a provider with the prognosis of liver disease. For example, based on the extent of liver damage, the provider can predict whether the disease (e.g. NAFL or NASH) will respond to treatment or mitigation efforts. Further, the biomarkers can indicate a patient's likelihood of responding favorably to surgical procedures such as bariatric surgery.

NAFL vs. Healthy Liver

Table 1A lists mRNAs of interest for detecting nonalcoholic fatty liver (NAFL). Levels of more than one of the mRNAs can be compared in a test patient to normal levels (i.e. those of subjects with healthy livers). These biomarkers are particularly beneficial for early screening of a liver disease. Similarly, corresponding proteins can be measured and used as biomarkers. Corresponding proteins are listed in Table 1B.

TABLE 1A
NAFL v. Healthy Liver

| | Affymetrix | | |
|---|---|---|---|
| No. | mRNA Probe ID | Description | Gene |
| 1 | 11753127_a_at | Aryl Hydrocarbon Receptor | ARNTL |
| | | Nuclear Translocator Like | |
| 2 | 11758490_s_at | Spartin | SPART |
| 3 | 11739116_s_at | EFR3 Homolog A | EFR3A |
| 4 | 11762833_x_at | mRNA/cDNA for LOC642846 | LOC642846 |
| | | gene (DEAD/H (Asp-Glu-Ala- | |
| | | Asp/His) box polypeptide | |
| | | 11-like), highly similar to | |
| | | Probable ATP-dependent | |
| | | RNA helicase DDX11 | |
| 5 | 11717380_x_at | FGFR1OP N-Terminal Like | FOPNL |
| 6 | 11730494_x_at | Potassium Two Pore Domain | KCNK2 |
| | | Channel Subfamily K | |
| | | Member 2 | |
| 7 | 11746418_a_at | Vitrin | VIT |
| 8 | 11722500_a_at | LysM Domain Containing 3 | LYSMD3 |
| 9 | 11737591_a_at | MybLike, SWIRM And MPN | MYSM1 |
| | | Domains 1 | |
| 10 | 11724280_a_at | Inhibitor of Growth Family | ING3 |
| | | Member 3 | |
| 11 | 11723439_at | Sushi Repeat Containing | SRPX2 |
| | | Protein X-Linked 2 | |
| 12 | 11756516_a_at | Phosphatidylserine | PISD |
| | | Decarboxylase | |
| 13 | 11738195_a_at | Fibroblast Growth Factor 1 | FGF1 |
| 14 | 11740395_a_at | Fibroblast Growth Factor | FGFR2 |
| | | Receptor 2 | |

_at = all the probes hit one known transcript;
_a = all probes in the set hit alternate transcripts from the same gene;
_s = all probes in the set hit transcripts from different genes;
_x = some probes hit transcripts from different genes.

NAFL vs. NASH

Conventional liver tests are generally ineffective in monitoring the progression of liver disease. Common liver function tests (LFTs) and imaging tests can only indicate an impairment in liver function. Because fatty liver (“NAFL”) often progresses to nonalcoholic steatohepatitis (“NASH”), a method of distinguishing between the two conditions can indicate progress of the disease. Thus, another embodiment is a method of detecting progression of liver disease. The biomarkers can be used in diagnosing and distinguishing NAFL (i.e. early stage liver disease) from NASH.

The severity of NAFLD can be described among four stages. Stage 1 is characterized by simple fatty liver (i.e. NAFL or hepatic steatosis). Fat begins to accumulate in individual cells but liver function is normal. There are usually no symptoms and patients may not realize they have the condition. Although the fat deposits are considered harmless, it is important that to prevent the disease from progressing to the next stage.

Stage 2 is referred to as nonalcoholic steatohepatitis (NASH). NASH is a more aggressive form of the condition, where the liver has become inflamed. Inflammation is the body's healing response to damage or injury and, in this case, is a sign that liver cells have become damaged. A person with NASH may have a dull or aching pain felt in the top right of their abdomen (over the lower right side of their ribs). Although liver function remains normal, NASH can be diagnosed with liver function tests.

Stage 3 is characterized by fibrosis. In this stage, there is persistent inflammation in the liver that results in the generation of fibrous scar tissue around the liver cells and blood vessels. This fibrous tissue replaces the healthy liver tissue. Typically, there is still enough healthy tissue for the liver to continue to function normally in stage 3. If fibrosis progresses, the patient can reach stage 4 of liver disease.

Stage 4 is characterized by cirrhosis. At this most severe stage, bands of scar tissue and clumps of liver cells develop. The liver shrinks and becomes lumpy which is known as cirrhosis. Cirrhosis progresses slowly gradually causing the liver to stop functioning. The damage caused by cirrhosis is irreversible and the patient may experience signs of liver failure. Cirrhosis tends to occur after the age of 50, usually after years of liver inflammation associated with the early stages of the disease. People with cirrhosis of the liver caused by NAFLD often also have type 2 diabetes.

Table 2A lists mRNAs of particular interest for diagnosing nonalcoholic fatty liver (NAFL) in comparison to non-alcoholic steatohepatitis (NASH). Similarly, corresponding proteins can be measured and used as biomarkers. Corresponding proteins are listed in Table 2B.

TABLE 2A
NAFL v. NASH

| | Affymetrix | | |
|---|---|---|---|
| No. | mRNA Probe ID | Description | Gene |
| 1 | 11733218_at | Transcription Factor AP-4 | TFAp4 |
| 2 | 11723753_a_at | Zinc Finger AN1-Type Containing 2A | ZFAND2A |
| 3 | 11722163_x_at | Polo Like Kinase 2 | PLK2 |
| 4 | 11758103_s_at | Transmembrane P24 Trafficking | TMED3 |
| | | Protein 3 | |
| 5 | 11724489_s_at | Aldo-Keto Reductase Family 1 | AKR1B10 |
| | | Member B10 | |
| 6 | 11732809_a_at | Prokineticin 2 | PROK2 |
| 7 | 11723106_a_at | Neutrophil Cytosolic Factor 4 | NCF4 |
| 8 | 11747524_a_at | Galactosylceramidase | GALC |
| 9 | 11732727_a_at | SLIT And NTRK Like Family | SLITRK4 |
| | | Member 4 | |

NASH vs. Healthy Liver

Table 3A lists mRNAs of interest for diagnosing NASH. Levels of more than one of the mRNAs can be compared in a test patient to normal levels (i.e. those of subjects with healthy livers). Similarly, corresponding proteins can be measured and used as biomarkers. Corresponding proteins are listed in Table 3B.

TABLE 3A
NASH v. Healthy Liver

| | Affymetrix | |
|---|---|---|
| | mRNA | |
| No. | Probe ID | Description |
| 1 | 16665621 | mRNA: CACHD1 gene |
| 2 | 16669169 | mRNA: CD2 molecule, transcript variant 2 |
| 3 | 16796773 | chr14: 101798302-101801206 |
| 4 | 16829985 | mRNA: ENO3 gene |
| 5 | 16886466 | chr2: 151026010-151157594 |
| 6 | 16890891 | mRNA: VIL1 gene |
| 7 | 16754913 | chr12: 88210263-88211608 |
| 8 | 17048083 | chr7: 89796904-89870091 |
| 9 | 16766132 | apolipoprotein F (APOF), mRNA |
| 10 | 16673227 | chr1: 164608802-164608908 |
| 11 | 16818272 | chr16: 31366509-31394318 |
| 12 | 16977052 | C-X-C motif chemokine ligand 10 (CXCL10), |
| | | mRNA |
| 13 | 16706008 | nudixhydrolase 13 (NUDT13), transcript variant 1, |
| | | mRNA |
| 14 | 16834015 | Rap guanine nucleotide exchange factor like 1 |
| | | (RAPGEFL1), transcript variant 3, mRNA |
| 15 | 17106795 | SH2 domain containing 1A (SH2D1A), transcript |
| | | variant 1, mRNA |
| 16 | 16714880 | chr10: 69556048-69597937 |
| 17 | 16851866 | chr18: 32073254-32471808 |
| 18 | 16830607 | sex hormone binding globulin (SHBG), transcript |
| | | variant 1, mRNA |
| 19 | 16659443 | Prame Family Member 17 (PRAMEF17), mRNA |
| 20 | 16660810 | RCAN Family Member 3, (RCAN3), mRNA |
| 21 | 16669963 | G Protein-Coupled Receptor 89A (GPR89A), mRNA |
| 22 | 16745651 | Olfactory Receptor Family 8 Subfamily B |
| | | Member 8 (OR8B8), mRNA |
| 23 | 16811975 | Tetraspanin3 (TSPAN3), mRNA |
| 24 | 16854594 | GRB2 Associated Regulator Of MAPK1 Subtype 1 |
| | | (GAREM1), mRNA |
| 25 | 16903953 | Activin A Receptor Type 1C (ACVR1C), mRNA |
| 26 | 16934434 | Apolipoprotein L3 (APOL3), mRNA |
| 27 | 17031373 | Ubiquitin D (FAT10), mRNA |
| 28 | 16909401 | Solute Carrier Family 16 Member 14 (SLC16A14), |
| | | mRNA |
| 29 | 17108996 | Family with Sequence Similarity 9 Member B |
| | | (FAM9B), mRNA |
| 30 | 17020964 | Interphotoreceptor Matrix Proteoglycan 1(IMPG1), |
| | | mRNA |
| 31 | 16977045 | C-X-C Motif Chemokine Ligand 9 (CXCL9), mRNA |
| 32 | 16677407 | SET And MYND Domain Containing 2 (SMYD2), |
| | | mRNA |
| 33 | 16695535 | Rho GTPase Activating Protein 30 (ARHGAP30), |
| | | mRNA |
| 34 | 16672654 | SLAM Family Member 7 (SLAMF7), mRNA |
| 35 | 16859395 | Myosin IXB (MYO9B), mRNA |
| 36 | 16661149 | CD52 molecule (CD52), mRNA |
| 37 | 17074914 | Macrophage Scavenger Receptor 1 (MSR1), a |
| | | transcript of gene ENSG00000038945.15 |

NASH vs. Hepatitis B vs. Hepatitis C vs. Healthy Liver

Common LFTs measure impaired liver function without a means of indicating a cause or particular liver disease. Another embodiment is a method of distinguishing a healthy liver from a liver with NASH, hepatitis B or hepatitis C. Table 4 lists miRNAs of particular interest for diagnosing NASH, Hepatitis B and Hepatitis C based on a comparison of levels in a healthy liver.

TABLE 4
NASH v. Hepatitis B v. Hepatitis C v. Healthy Liver miRNA

| No. | Reference | Description |
|---|---|---|
| 1 | hsa-let-7d | Homo sapiens Let-7d stem-loop |
| 2 | hsa-miR-149 | Homo sapiens miR-149 stem-loop |
| 3 | hsa-miR-513a-3p | Homo sapiens miR-513a-1 stem-loop |
| 4 | hsa-miR-192 | Homo sapiens miR-192 stem-loop |
| 5 | hsa-miR-23b | Homo sapiens miR-23 stem-loop |
| 6 | hsa-miR-301a | Homo sapiens miR-301a stem-loop |
| 7 | hsa-miR-933 | Homo sapiens miR-933 stem-loop |
| 8 | hsa-miR-148a | Homo sapiens miR-148a stem-loop |
| 9 | hsa-miR-17 | Homo sapiens miR-17 stem-loop |
| 10 | hsa-miR-423-3p | Homo sapiens miR-423 stem-loop |
| 11 | hsa-miR-563 | Homo sapiens miR-563 stem-loop |
| 12 | hsa-miR-596 | Homo sapiens miR-596 stem-loop |
| 13 | hsa-miR-150 | Homo sapiens miR-150 stem-loop |
| 14 | hsa-miR-1260 | Homo sapiens miR-1260 stem-loop |
| 15 | hsa-miR-1539 | Homo sapiens miR-1539 stem-loop |
| 16 | hsa-miR-15b | Homo sapiens miR-15b stem-loop |
| 17 | hsa-miR-887 | Homo sapiens miR-887 stem-loop |
| 18 | hsa-miR-602 | Homo sapiens miR-602 stem-loop |
| 19 | hsa-miR-610 | Homo sapiens miR-610 stem-loop |
| 20 | hsa-miR-1237 | Homo sapiens miR-1237 stem-loop |
| 21 | hsa-miR-20b | Homo sapiens miR-20b stem-loop |
| 22 | hsa-miR-425 | Homo sapiens miR-425 stem-loop |
| 23 | hsa-miR-718 | Homo sapiens miR-718 stem-loop |
| 24 | hsa-miR-595 | Homo sapiens miR-595 stem-loop |
| 25 | hsa-miR-101 | Homo sapiens miR-101 stem-loop |
| 26 | hsa-miR-483-3p | Homo sapiens miR-483 stem-loop |
| 27 | hsa-miR-103 | Homo sapiens miR-103 stem-loop |
| 28 | hsa-miR-186 | Homo sapiens miR-186 stem-loop |
| 29 | hsa-miR-33b | Homo sapiens miR-33b stem-loop |
| 30 | hsa-miR-1225-3p | Homo sapiens miR-1225 stem-loop |
| 31 | hsa-miR-331-3p | Homo sapiens miR-331 stem-loop |
| 32 | hsa-miR-215 | Homo sapiens miR-215 stem-loop |

The methods and materials can be used for assessing subjects (e.g., human patients) for liver disease. For example, embodiments include materials and methods for using identifiable markers to assist clinicians in assessing stages of liver disease, assessing the likelihood of response and outcomes of therapy, and predicting long-term disease outcomes. Further, subjects with liver disease can be diagnosed based on the presence of certain diagnostic indicators in plasma from the subject. The technology provides diagnostic methods for predicting and/or prognosticating the effectiveness of treatment. In particular, the subject technology concerns the diagnosis of liver disease based on one or more combinations of markers.

Multiple mRNA/miRNA biomarkers can be used from a single serum sample taken from a subject. According to some embodiments, multiple biomarkers are assessed and measured from different samples taken from the patient. According to some embodiments, the subject technology is used for a kit for predicting, diagnosing or monitoring responsiveness of a liver disease treatment or therapy, wherein the kit is calibrated to measure marker levels in a sample from the patient.

According to some embodiments, the amount of biomarkers can be determined by using, for example, a reagent that specifically binds with the biomarker protein or a fragment thereof, (e.g., an antibody, a fragment of an antibody, or an antibody derivative). The level of expression can be determined using a method common in the art such as proteomics, flow cytometry, immunocytochemistry, immunohistochemistry, enzyme-linked immunosorbent assay, multi-channel enzyme linked immunosorbent assay, and variations thereof. The expression level of a biomarker in the biological sample can also be determined by detecting the level of expression of a transcribed biomarker polynucleotide or fragment thereof encoded by a biomarker gene, which may be cDNA, mRNA or heterogeneous nuclear RNA (hnRNA). The step of detecting can include amplifying the transcribed biomarker polynucleotide, and can use the method of quantitative reverse transcriptase polymerase chain reaction. The expression level of a biomarker can be assessed by detecting the presence of the transcribed biomarker polynucleotide or a fragment thereof in a sample with a probe which anneals with the transcribed biomarker polynucleotide or fragment thereof under stringent hybridization conditions.

Also provided herein are compositions and kits for practicing the methods. For example, in some embodiments, reagents (e.g., primers, probes) specific for one or more markers are provided alone or in sets (e.g., sets of primers pairs for amplifying a plurality of markers). Additional reagents for conducting a detection assay can also be provided (e.g., enzymes, buffers, positive and negative controls for conducting QuARTS, PCR, sequencing, bisulfite, or other assays). In some embodiments, the kits containing one or more reagent necessary, sufficient, or useful for conducting a method are provided. Also provided are reactions mixtures containing the reagents. Further provided are master mix reagent sets containing a plurality of reagents that may be added to each other and/or to a test sample to complete a reaction mixture.

In some embodiments, the technology described herein is associated with a programmable machine designed to perform a sequence of arithmetic or logical operations as provided by the methods described herein. For example, some embodiments of the technology are associated with (e.g., implemented in) computer software and/or computer hardware. In one aspect, the technology relates to a computer comprising a form of memory, an element for performing arithmetic and logical operations, and a processing element (e.g., a microprocessor) for executing a series of instructions (e.g., a method as provided herein) to read, manipulate, and store data. Therefore, certain embodiments employ processes involving data stored in or transferred through one or more computer systems or other processing systems. Embodiments also relate to apparatus for performing these operations. This apparatus can be specially constructed for the required purposes, or it can be a general-purpose computer (or a group of computers) selectively activated or reconfigured by a computer program and/or data structure stored in the computer. In some embodiments, a group of processors performs some or all of the recited analytical operations collaboratively (e.g., via a network or cloud computing) and/or in parallel.

In some embodiments, a microprocessor is part of a system for determining the presence of one or more mRNA or miRNA (labeled herein as hsa-miR or has-miRs) associated with a liver disease; generating standard curves; determining a specificity and/or sensitivity of an assay or marker; calculating an ROC curve; sequence analysis; all as described herein or is known in the art.

In some embodiments, a microprocessor is part of a system for determining the amount, such as concentration, of one or more mRNA or miRNA associated with a liver disease; generating standard curves; determining a specificity and/or sensitivity of an assay or marker; calculating an ROC curve; sequence analysis; all as described herein or is known in the art. The amount of one or more mRNA or miRNA can be determined by abundance, measured per mole or millimole. The amount of mRNA or miRNA can be determined by fluorescence, other measurement using an optical signal or other measurement known to one of skill to measure an mRNA or miRNA.

In some embodiments, a microprocessor or computer uses an algorithm to measure the amount of an mRNA or miRNA. The algorithm can include a mathematical interaction between a marker measurement or a mathematical transform of a marker measurement. The mathematical interaction and/or mathematical transform can be presented in a linear, nonlinear, discontinuous or discrete manner.

In some embodiments, a software or hardware component receives the results of multiple assays and determines a single value result to report to a user that indicates a liver disease risk based on the results of the multiple assays. Related embodiments calculate a risk factor based on a mathematical combination (e.g., a weighted combination, a linear combination) of the results from multiple assays as disclosed herein.

Some embodiments comprise a storage medium and memory components. Memory components (e.g., volatile and/or nonvolatile memory) find use in storing instructions (e.g., an embodiment of a process as provided herein) and/or data (e.g., a work piece such as methylation measurements, sequences, and statistical descriptions associated therewith). Some embodiments relate to systems also comprising one or more of a CPU, a graphics card, and a user interface (e.g., comprising an output device such as display and an input device such as a keyboard). Programmable machines associated with the technology comprise conventional extant technologies and technologies in development or yet to be developed (e.g., a quantum computer, a chemical computer, a DNA computer, an optical computer, a spintronics based computer, etc.).

In some embodiments, the technology comprises a wired (e.g., metallic cable, fiber optic) or wireless transmission medium for transmitting data. For example, some embodiments relate to data transmission over a network (e.g., a local area network (LAN), a wide area network (WAN), an ad-hoc network, the internet, etc.). In some embodiments, programmable machines are present on such a network as peers and in some embodiments the programmable machines have a client/server relationship. In some embodiments, data are stored on a computer-readable storage medium such as a hard disk, flash memory, memory stick, optical media, a floppy disk, etc.

In some embodiments, the technology provided herein is associated with a plurality of programmable devices that operate in concert to perform a method as described herein. For example a plurality of computers (e.g., connected by a network) can work in parallel to collect and process data, e.g., in an implementation of cluster computing or grid computing or some other distributed computer architecture that relies on complete computers (with onboard CPUs, storage, power supplies, network interfaces, etc.) connected to a network (private, public, or the internet) by a conventional network interface, such as Ethernet, fiber optic, or by a wireless network technology.

For example, some embodiments provide a computer that includes a computer-readable medium. The embodiment includes a random access memory (RAM) coupled to a processor. The processor executes computer-executable program instructions stored in memory. Such processors may include a microprocessor, an ASIC, a state machine, or other processor, and can be any of a number of computer processors, such as processors from Intel Corporation of Santa Clara, Calif. and Motorola Corporation of Schaumburg, Ill. Such processors include, or may be in communication with, media, for example computer-readable media, which stores instructions that, when executed by the processor, cause the processor to perform the steps described herein.

Embodiments of computer-readable media include, but are not limited to, an electronic, optical, magnetic, or other storage or transmission device capable of providing a processor with computer-readable instructions. Other examples of suitable media include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. The instructions may comprise code from any suitable computer-programming language, including, for example, C, C++, C #, Visual Basic, Java, Python, Perl, and JavaScript.

Computers are connected in some embodiments to a network. Computers may also include a number of external or internal devices such as a mouse, a CD-ROM, DVD, a keyboard, a display, or other input or output devices. Examples of computers are personal computers, digital assistants, personal digital assistants, cellular phones, mobile phones, smart phones, pagers, digital tablets, laptop computers, internet appliances, and other processor-based devices. In general, the computers related to aspects of the technology provided herein may be any type of processor-based platform that operates on any operating system, such as Microsoft Windows, Linux, UNIX, Mac OS X, etc., capable of supporting one or more programs comprising the technology provided herein. Some embodiments comprise a personal computer executing other application programs (e.g., applications). The applications can be contained in memory and can include, for example, a word processing application, a spreadsheet application, an email application, an instant messenger application, a presentation application, an Internet browser application, a calendar/organizer application, and any other application capable of being executed by a client device.

All such components, computers, and systems described herein as associated with the technology may be logical or virtual. It is also envisioned that embodiments could be accomplished as computer signals embodied in a carrier wave, as well as signals (e.g. electrical and optical) propagated through a transmission medium. Thus, the various types of information discussed above could be formatted in a structure, such as a data structure, and transmitted as an electrical signal through a transmission medium or stored on a computer readable medium.

In some embodiments, the disclosure provides a system for predicting progression of a liver disease. In another embodiment a liver disease is NAFL, NASH, hepatitis B or hepatitis C. In an embodiment, a liver disease, including any of the aforementioned diseases can be identified and the particular disease predicted in a patient, the system comprising: an apparatus configured to determine expression levels of nucleic acids, proteins, peptides or other molecule from a biological sample taken from the individual; and hardware logic designed or configured to perform operations comprising: (a) receiving expression levels of a collection of signature genes from a biological sample taken from said individual, wherein said collection of signature genes comprises at least two genes selected from the group consisting of the sequences set forth in Table 1A, 2A or 3A or the mi-RNAs set forth in Table 4A.

Information relevant to the patient's diagnosis include, but are not limited to, age, ethnicity, pertinent past medical history related to co-morbidity, other history such as exposure to chemicals, alcohol/drug use, family history of liver disease, physical exam findings, radiological findings, biopsy date, biopsy result, swelling of the abdomen and legs, bruising easily, changes in the color of your stool and urine, and jaundice, or yellowing of the skin and eyes, local vs. distant disease recurrence and survival outcome. These clinical variables may be included in the predictive model in various embodiments.

In an embodiment, a biomarker or biomarker panel is selected, a method for diagnosing an individual that may be suffering from a liver disease such as NAFL, NASH, hepatitis B or hepatitis C and can comprise one or more of the following steps: 1) collect or otherwise obtain a biological sample; 2) perform an analytical method to detect and measure the biomarker or biomarkers in the panel in the biological sample; 3) perform any data normalization or standardization required for the method used to collect biomarker values; 4) calculate a biomarker score; 5) combine the biomarker scores to obtain a total diagnostic score; and 6) report the individual's diagnostic score. This method of diagnosis can be conducted using a computer and software programs for analysis of data collected from nucleic acid, protein, peptide or other biological molecules. In this approach, the diagnostic score may be a single number determined from the sum of all the marker calculations that is compared to a preset threshold value that is an indication of the presence or absence of disease. Or the diagnostic score can be a series of bars that each represent a biomarker value and the pattern of the responses may be compared to a pre-set pattern for determination of the presence or absence of disease.

For both DNA and RNA, the nucleic acid can be isolated from plasma or blood sample. The DNA or RNA can be extracellular or extracted from a cell in the plasma or blood sample. The DNA or RNA can also be extracted from a cellular biopsy (i.e. live biopsy). For a protein or peptide or other biological molecule, such can be isolated from plasma or a blood sample. The protein or peptide or other biological molecule can be extracellular or extracted from a cell in the plasma or a blood sample. The protein or peptide or other biological molecule can also be extracted from a cellular biopsy (i.e. live biopsy).

It is also noted that many of the structures, materials, and acts recited herein can be recited as means for performing a function or step for performing a function. Therefore, it should be understood that such language is entitled to cover all such structures, materials, or acts disclosed within this specification and their equivalents, including the matter incorporated by reference.

The liver disease, including NAFL, NASH, hepatitis B or hepatitis C biomarker analysis system can provide functions and operations to complete data analysis, such as data gathering, processing, analysis, reporting and/or diagnosis. For example, in one embodiment, the computer system can execute the computer program that may receive, store, search, analyze, and report information relating to the liver disease biomarkers. The computer program can comprise multiple modules performing various functions or operations, such as a processing module for processing raw data and generating supplemental data and an analysis module for analyzing raw data and supplemental data to generate a liver disease status and/or diagnosis. Diagnosing liver disease status may comprise generating or collecting any other information, including additional biomedical information, regarding the condition of the individual relative to the disease, identifying whether further tests may be desirable, or otherwise evaluating the health status of the individual.

The liver disease biomarker analysis system can provide functions and operations to complete data analysis, such as data gathering, processing, analysis, reporting and/or diagnosis. For example, in one embodiment, the computer system can execute the computer program that may receive, store, search, analyze, and report information relating to the liver disease biomarkers. The computer program may comprise multiple modules performing various functions or operations, such as a processing module for processing raw data and generating supplemental data and an analysis module for analyzing raw data and supplemental data to generate a liver disease status and/or diagnosis. Diagnosing liver disease can comprise generating or collecting any other information, including additional biomedical information, regarding the condition of the individual relative to the disease, identifying whether further tests may be desirable, or otherwise evaluating the health status of the individual.

As used herein, a “computer program product” refers to an organized set of instructions in the form of natural or programming language statements that are contained on a physical media of any nature (e.g., written, electronic, magnetic, optical or otherwise) and that may be used with a computer or other automated data processing system. Such programming language statements, when executed by a computer or data processing system, cause the computer or data processing system to act in accordance with the particular content of the statements. Computer program products include without limitation: programs in source and object code and/or test or data libraries embedded in a computer readable medium. Furthermore, the computer program product that enables a computer system or data processing equipment device to act in pre-selected ways may be provided in a number of forms, including, but not limited to, original source code, assembly code, object code, machine language, encrypted or compressed versions of the foregoing and any and all equivalents.

In one embodiment, a computer program product is provided for indicating a likelihood of liver disease including NAFL, NASH, hepatitis B or hepatitis C. The computer program product includes a computer readable medium embodying program code executable by a processor of a computing device or system, the program code comprising: code that retrieves data attributed to a biological sample from an individual, wherein the data comprises biomarker values that each correspond to one of at least N biomarkers in the biological sample selected from the group of biomarkers provided in Table 1A, 1B, 2A, 2B, 3A, 3B or 4; and code that executes a classification method that indicates a liver disease status of the individual as a function of the biomarker values.

In yet another embodiment, a computer program product is provided for indicating a likelihood of liver disease including NAFL, NASH, hepatitis B or hepatitis C. The computer program product includes a computer readable medium embodying program code executable by a processor of a computing device or system, the program code comprising: code that retrieves data attributed to a biological sample from an individual, wherein the data comprises a biomarker value corresponding to a biomarker in the biological sample selected from the group of biomarkers provided in Table 1A, 1B, 2A, 2B, 3A, 3B or 4; and code that executes a classification method that indicates a liver disease status of the individual as a function of the biomarker value.

The kit (i.e. diagnostic kit) can include reagents for determining, from a plasma sample of a subject, the amount of mRNAs/miRNAs or mutations in a gene based on assaying the nucleic acids, proteins, peptides or other biological molecule isolated from a diseased liver cell circulating cell or the remnants of a circulating cell present in plasma, including a protein, peptide or other biological molecule. The nucleic acid can be a deoxyribonucleic acid (DNA), a ribonucleic acid (RNA) and/or an artificial nucleic acid, including an artificial nucleic acid analogue. Along with miRNAs, other RNAs include non-coding RNA (ncRNA), transfer RNA (tRNA), messenger RNA (mRNA), small interfering RNA (siRNA), piwi RNA (piRNA), small nuclear RNA (snoRNA), small nuclear (snRNA), extracellular RNA (exRNA), and ribosomal RNA (rRNA).

The disclosed methods and assays provide for convenient, efficient, and potentially cost-effective means to obtain data and information useful in assessing appropriate or effective therapies for treating patients. The kit can use conventional methods for detecting the biomarkers, whether a protein, peptide, other biological molecule or an RNA or a DNA to be assessed include protocols that examine the presence and/or expression of a desired nucleic acid, for example a SNP, in a sample. Tissue or cell samples from mammals can be conveniently assayed for, e.g., genetic-marker RNA, including in an embodiment an miRNA or DNAs using Northern, dot-blot, or polymerase chain reaction (PCR) analysis, array hybridization, RNase protection assay, or using DNA SNP chip microarrays, which are commercially available, including DNA micro array snapshots. For example, real-time PCR (RT-PCR) assays such as quantitative PCR assays are well known in the art.

Probes used for PCR can be labeled with a detectable marker, such as, for example, a radioisotope, fluorescent compound, bioluminescent compound, a chemiluminescent compound, metal chelator, or enzyme. Such probes and primers can be used to detect the presence of a mutation in a DNA, an RNA and in one embodiment, an miRNA in a sample and as a means for detecting a cell expressing the miRNA. As will be understood by the skilled artisan, a great many different primers and probes can be prepared based on known sequences and used effectively to amplify, clone, and/or determine the presence and/or levels of mRNAs/miRNAs.

The biomarkers are particularly useful in diagnosing liver disease as their expression patterns are different when comparing healthy subjects with subjects that have liver disease. The expression of biomarkers typically includes both up- and down-regulated levels of miRNAs.

In an embodiment, the biomarkers set forth herein can determine if a patient has liver disease or does not have liver disease. In an embodiment, the biomarkers can identify the type of liver disease and/or the progress of the disease. In an embodiment, the biomarkers can identify the extent of liver damage, the likelihood of recovery and preferred methods of treatment/drugs. In another embodiment, the biomarkers can identify the liver disease as one or more of hepatitis A, hepatitis B, hepatitis C, hepatitis E, autoimmune hepatitis, fatty liver disease (FLD), NAFL, NASH, cirrhosis, liver cancer, hemochromatosis, Wilson's disease and conditions that affect the veins of the liver, such as Budd-Chiari syndrome. In on embodiment, multiple tests can be conducted to diagnose a particular liver disease. For example, the biomarkers of Table 3 can be used in a first test, followed by the markers of Table 4 for a second test. In another example, the biomarkers of Table 1A can be used in a first test, followed by the markers of Table 2A for a second test. In yet another example, the biomarkers of Table 3A can be used in a first test, followed by the markers of Table 2A for a second test.

The biomarkers described herein can be detected in DNA, including in an embodiment, one or mutations associated with a region of a gene, a snp or one or mutation on one or more chromosomes. The biomarkers described herein can also be detected in an RNA, including in an embodiment, an miRNA, a tRNA, an mRNA or other form of RNA.

Other methods for determining the level of the biomarker besides RT-PCR or another PCR-based method include proteomics techniques, as well as individualized genetic profiles. Individualized genetic profiles can be used to detect or diagnose a liver disease based on patient response at a molecular level. The specialized microarrays herein, (e.g., oligonucleotide microarrays or cDNA microarrays) can include one or more biomarkers having expression profiles that correlate with either sensitivity or resistance to one or more antibodies.

A nucleic acid sample can be obtained by any method known in the art. One of skill in the art will appreciate that it is desirable to have nucleic samples containing target nucleic acid sequences that reflect the transcripts of interest. Therefore, suitable nucleic acid samples may contain transcripts of interest. Suitable nucleic acid samples, however, may contain nucleic acids derived from the transcripts of interest. As used herein, a nucleic acid derived from a transcript refers to a nucleic acid for whose synthesis the mRNA transcript or a subsequence thereof has ultimately served as a template. Thus, a cDNA reverse transcribed from a transcript, an RNA transcribed from that cDNA, a DNA amplified from the cDNA, an RNA transcribed from the amplified DNA, etc., are all derived from the transcript and detection of such derived products is indicative of the presence and/or abundance of the original transcript in a sample. Thus, suitable samples include transcripts of the gene or genes, cDNA reverse transcribed from the transcript, cRNA transcribed from the cDNA, DNA amplified from the genes, RNA transcribed from amplified DNA, and the like. Transcripts, as used herein, can include pre-mRNA nascent transcript(s), transcript processing intermediates, mature mRNA(s) and degradation products. It is not necessary to monitor all types of transcripts to practice this invention. For example, one may choose to practice the invention to measure the mature mRNA levels only.

Each of the mRNAs, miRNAs and genes described above, would be well known to the person of skill in the art, as would their encoded protein expression products. In general, the gene from which the RNA transcripts are identifiable by the Affymetrix probes are well known and characterized genes. However, to the extent that some of the probes detect RNA transcripts which are not yet defined, these genes are indicated as “the gene or genes detected by Affymetrix probe x.” In some cases a number of genes may be detectable by a single probe. This is also indicated where appropriate. It should be understood, however, that this is not intended as a limitation as to how the expression level of the subject gene can be detected. In the first case, it would be understood that the subject gene transcript is also detectable by other probes which would be present on an Affymetrix gene chip. The reference to a single probe is merely included as an identifier of the gene transcript of interest. In terms of actually screening for the transcript, however, one can use a probe directed to any region of the transcript and not just to the terminal 600 bp transcript region to which the Affymetrix probes are generally directed.

Reference to each of the genes detailed above and their transcribed and translated expression products should therefore be understood as a reference to all forms of these molecules and to fragments, mutants or variants thereof. As would be appreciated by the person of skill in the art, some genes are known to exhibit allelic variation between individuals. Accordingly, the present invention should be understood to extend to such variants which, in terms of the present diagnostic applications, achieve the same outcome despite the fact that minor genetic variants between the actual nucleic acid sequences may exist between individuals. The present invention should therefore be understood to extend to all RNA (eg mRNA, primary RNA transcript, miRNA, tRNA, rRNA etc), cDNA and peptide isoforms which arise from alternative splicing or any other mutation, polymorphic or allelic variation. It should also be understood to include reference to any subunit polypeptides such as precursor forms which may be generated, whether existing as a monomer, multimer, fusion protein or other complex.

While a single biomarker can provide evidence of liver disease, reliability and accuracy is improved when multiple biomarkers are used. Thus, in embodiments, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 biomarkers can be used. The one biomarker or a combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 and/or 38 biomarkers can be stored in a liquid or in a dry form, including, following lyophilization. If the one biomarker or a combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 and/or 38 biomarkers are stored dry, the one biomarker or a combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 and/or 38 biomarkers can be resuspended using water or a solution one of skill in the art would know would know would result in the stable resuspension of the one biomarker or a combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 and/or 38 biomarkers.

Method of Diagnoses Using mRNAs as Biomarkers

Detection and quantification of expressed mRNA/miRNA can use standard techniques known to one skilled in the art. For example, the expression or amount of a particular mRNA in the body fluid sample or in the tissue specimen, or in a liver biopsy sample or a liver tissue sample identified, is determined by immunohistochemical (IHC) methods, by immunofluorescence (IF) methods, by RNA in-situ hybridization, by reverse transcriptase polymerase chain reaction (RTPCR), especially quantitative real time RT-PCR (qRT-PCR), or by a combination of these methods. Other methods for determining the level of (expression of) a particular mRNA include MALDI-MS (including surface enhanced laser desorption/ionization mass spectrometry (SELDI-MS), especially surface-enhanced affinity capture (SEAC), surface-enhanced need desorption (SEND) or surface-enhanced photo label attachment and release (SEPAR) for the determination of proteins in samples for diagnosing and staging of steatohepatitis), antibody testing (including immunoprecipitation, Western blotting, Enzyme-linked immuno sorbent assay (ELISA), Enzyme-linked immuno sorbent assay (RIA), dissociation-enhanced lanthanide fluoro immuno assay (DELFIA), scintillation proximity assay (SPA) for diagnosing, staging or monitoring steatohepatitis samples under investigation of specific marker proteins), and quantitative nucleic acid testing, especially PCR, LCR and RT-PCR of samples for marker (KRT23) mRNA detection and quantification.

Preferably the difference in protein amount or in expressed mRNA for a biomarker is at least 5%, 10% or 20%, more preferred at least 50% or may even be as high as 75% or 100%. More preferred this difference in the level of expression or protein amount is at least 200%, i.e. two-fold, at least 500%, i.e. five-fold, or at least 1000%, i.e. 10-fold. The expression level for a biomarker according to the present invention expressed lower or higher in a disease sample than in a healthy, normal (e.g. NAFL) sample is at least 5%, 10% or 20%, more preferred at least 50% or may even be 75% or 100%, i.e. two-fold higher, preferably at least ten-fold higher in the disease (e.g. NAFL) sample. Whether a biomarker level is increased in a given detection method can be established by analysis of a multitude of disease samples with the given detection method. This can then form a suitable level from which the “increased” status can be determined; e.g. by the above % difference or -fold change. A changed level of expression of a biomarker can be indicative of liver disease, for example steatohepatitis. In some cases, no biomarker expression is detectable in healthy samples. In such case, any detection with normal test systems is already an “increased” level within the meaning of the present application.

One or more of the biomarkers can be used in a method of diagnosing liver disease or determining a prognosis of a test subject with liver disease. In this manner, one biomarker or a combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 biomarkers can be used in a method of diagnosing liver disease or determining a prognosis of a test subject with liver disease. In this manner, at least one biomarker or a combination of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 biomarkers can be used in a method of diagnosing liver disease or determining a prognosis of a test subject with liver disease.

In this manner, no more than one biomarker or a combination of no more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 biomarkers can be used in a method of diagnosing liver disease or determining a prognosis of a test subject with liver disease. In this manner, about one biomarker or a combination of about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 biomarkers can be used in a method of diagnosing liver disease or determining a prognosis of a test subject with liver disease.

In a first step, the expression levels of one or more miRNAs are measured in plasma samples from subjects with liver disease. In an embodiment, the expression levels of one biomarker or a combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 biomarkers can be used to generate a footprint or signature for subsequent diagnosis of patients. In an embodiment, the expression levels of at least one biomarker or a combination of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 biomarkers can be used to generate a footprint or signature for subsequent diagnosis of patients.

In an embodiment, the expression levels of no more than one biomarker or a combination of no more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 biomarkers can be used to generate a footprint or signature for subsequent diagnosis of patients. In an embodiment, the expression levels of about one biomarker or a combination of about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 biomarkers can be used to generate a footprint or signature for subsequent diagnosis of patients.

Next, expression levels of the same nucleic acids, including DNA and/or RNA and further including mRNAs are measured in plasma, blood or tissue samples from healthy subjects. This is used as a control. Thereafter, samples from healthy patients can be compared to identifying mRNAs that have altered levels of expression in the plasma samples from the subjects with liver disease. A biomarker fingerprint or signature can be created from the mRNAs with altered levels of expression. This can be used for diagnosing or determining the prognosis of liver disease in the test subject by comparing of levels of mRNAs from plasma of the test subject. Conventional statistical analysis can be used to determine, for example, confidence levels.

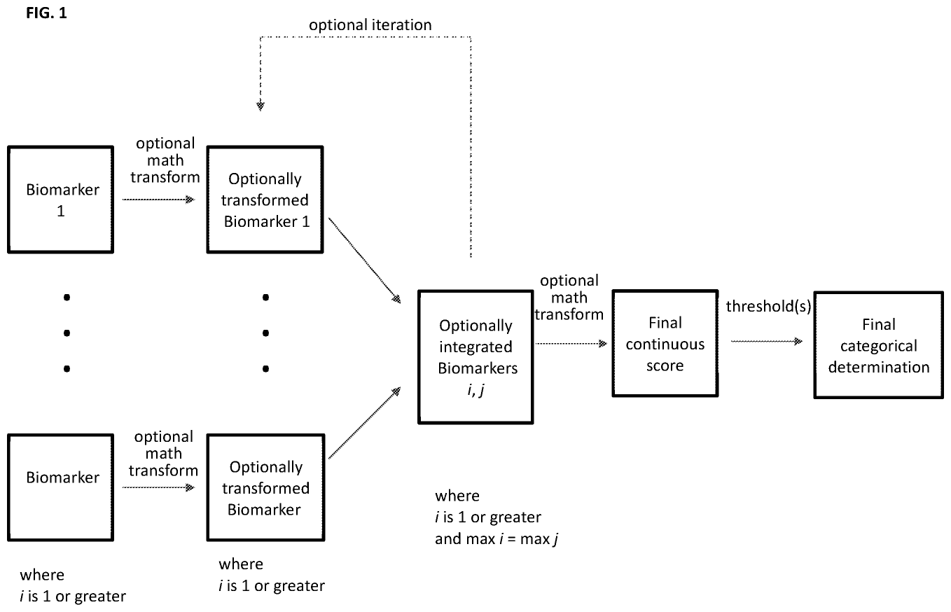

FIG. 1 depicts a method of combining results from biomarkers to achieve a final categorical determination. Multiple biomarkers can be measured in a patient. The results can be compiled to produce a single categorical determination according to the following steps.
1. For a patient or sample, on which was measured a set of biomarkers (each biomarker is bᵢ where i is at least 1).
2. Optionally transform each biomarker using a mathematical or logical operation.
3. For a subset of the i biomarkers with j biomarkers in the subset (at least 2 member biomarkers per set), optionally integrate the biomarker measures using a mathematical or logical operation (e.g. b₁/b₂) to form an “integrated biomarker”.
4. Optionally perform step 3 on k subsets.
5. Optionally iterate original biomarkers and integrated biomarkers through steps 2, 3, 4 to result in a final, continuous score.
6. Apply t thresholds (where t is 1 or greater) to categorize the patient or sample into a decision category (diagnostic, prognostic, treatment responder, etc.).

An alternative approach includes the following steps.
1. For a patient or sample, on which was measured a set of biomarkers (each biomarker is bᵢ where i is at least 1).
2. Mathematically transform each biomarker (the definition of transform may be no transform, e.g. no mathematical operation or a non-modifying operation such as multiplying by 1).
3. Optionally mathematically integrate from 2 to i of the biomarkers into a final score (e.g. by weights applied to each and results added, like linear regression, or by other algorithmic steps including iterative steps).
4. Using t thresholds (where t is at least 1), categorize the patient or sample into 1 of t+1 categories.

### EXAMPLES

The following non-limiting examples are provided for illustrative purposes only in order to facilitate a more complete understanding of representative embodiments now contemplated. These examples are intended to be a mere subset of all possible contexts in which the components of the formulation may be combined. Thus, these examples should not be construed to limit any of the embodiments described in the present specification, including those pertaining to the type and amounts of components of the formulation and/or methods and uses thereof.

### Example 1

### Early Detection of Fatty Liver (NAFL) Using Biomarkers

A pre-symptomatic diagnosis of patients with liver disease would be of great value, not only for a better understanding of the disease's pathophysiology, but also for providing early treatment and mitigation efforts. In this example, a patient with liver disease is asymptomatic. The patient is a 50-year old male who desires to be evaluated by a physician for the possibility of liver damage. The patient is borderline obese and has a history of drug use and high blood pressure. The patient also has a family history of liver disease. Because of these risk factors, his physician orders standard liver function tests (LFTs).

The results of the LFTs are within normal range. The patient is further screened using the biomarkers of Table 1A. The patient provides a sample (e.g. blood, plasma, urine or saliva) for biomarker analysis. The results indicate that the patient has NAFL and is in the early stages of liver disease. Based on these results, the patient is referred to specialist for further evaluation and instructed to make diet/lifestyle changes. The physician also recommends regular (i.e. annual) testing to ensure that the liver disease does not advance.

### Example 2

Distinguishing NAFL from NASH Using Biomarkers

In this example, a patient suffers from liver disease. Biomarkers are used to identify the progression of the disease. The four stages of the disease are stage 1: simple fatty liver (NAFL); stage 2: nonalcoholic steatohepatitis (NASH); stage 3: fibrosis and stage 4: cirrhosis. It can be particularly beneficial to accurately and reliably monitor progression of the disease, particularly between stage 1 and stage 2.

In this example, the patient has undergone standard liver function tests (LFTs). The results indicate the likelihood of liver disease. Further, the liver appears mildly inflamed when observed by ultrasound. The physician orders the biomarker test described herein (Table 2A) to determine the progress of the liver disease. The patient provides a sample for biomarker analysis. Specifically, the test determines progression from NAFL to NASH.

The test indicates that the patient has NAFL. As with the first example, the patient is referred to specialist for further evaluation and instructed to make diet/lifestyle changes. The physician also recommends regular (i.e. annual) testing to ensure that the liver disease does not advance.

### Example 3

### Detection of Liver Disease (NASH) Using Biomarkers

In this example, a patient has no symptoms of liver disease but undergoes evaluation because of risk factors. The patient's LFT's tests are mildly abnormal. Specifically, albumin levels are low and bilirubin levels are high. Further, the liver appears mildly inflamed when observed by ultrasound.

The physician orders the biomarker test described herein (Table 3A) to accurately diagnose the liver disease. The patient provides a sample for biomarker analysis. The test provides the physician with biomarker levels to diagnose the patient with NASH. As with the first example, the patient is referred to specialist for further evaluation and to discuss treatment options. Had the patient tested negative for NASH, additional evaluation would be pursued to rule out hepatitis B or C.

### Example 4

### Distinguishing Between NASH, Hepatitis B and Hepatitis C Using Biomarkers

In this example, a patient suffers from mild fatigue and jaundice. The patient's LFT's tests show low albumin levels and high bilirubin levels. Further, the liver appears mildly inflamed when observed by ultrasound.

The physician orders the biomarker test described herein (Table 4) to distinguish a healthy liver from one with NASH, hepatitis B or hepatitis C. Specifically, the test rules out hepatitis and positively confirms that the patient has NASH. As with the previous example, the patient is referred to specialist for further evaluation and to discuss treatment options.

### Example 5

### Kit for Rapid Screening of Liver Disease

The following working example is based on configurations described above. Embodiments of the invention can be compiled into a diagnostic kit for diagnosing liver disease. The kit can identify one or more target cells that have the biomarkers for liver disease in plasma from a test subject.

In this example, a patient is asymptomatic but is concerned because of risk factors (i.e. daily medication) and family history. Rather than standard liver function tests (LFTs), a health care provider uses the kit to measure the biomarkers of Table 1A. The test is administered to the patient annually to ensure that he has a healthy liver.

The kit can include a collection of nucleic acid molecules such that each nucleic acid molecule encodes a mRNA sequence. The nucleic acid molecules can be used to identify variations in expression levels of one or more mRNAs in a plasma sample from a test subject. The expression levels of the mRNAs can be used in a comparison/analysis of test samples with a fingerprint indicative of the presence of liver disease.

In certain embodiments, the present disclosure provides kits for diagnosing liver disease. In one embodiment, the liver disease is FLD or NASH. The kits can include one biomarker or a combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 biomarkers disclosed herein. The skilled artisan will appreciate that the number of biomarkers may be varied without departing from the nature of the present disclosure, and thus other combinations of biomarkers are also encompassed by the present disclosure. The skilled artisan will know which one biomarker or a combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 biomarkers to use based on the symptoms of the patient suffering from liver disease.

In a specific embodiment, a kit includes the one biomarker or a combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 biomarkers disclosed herein. In certain embodiments, the kit is for diagnosing liver disease. In another embodiment, the kit is for diagnosing an liver disease. The kit can further optionally include instructions for use. The kit can further optionally include (e.g., comprise, consist essentially of, consist of) tubes, applicators, vials or other storage container with the above mentioned biomarker and/or vials containing one or more of the biomarkers. In an embodiment, each biomarker is in its own tube, applicator, vial or storage container or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 biomarkers are in a tube, applicator, vial or storage container.

The kits, regardless of type, will generally include one or more containers into which the one biomarker or a combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 biomarkers are placed and, preferably, suitably aliquoted. The components of the kits may be packaged either in aqueous media or in lyophilized form.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

TABLE 1B
mRNA and Protein Biomarkers: NAFL v. Healthy Liver

| No. | Marker Probe ID | Description | Gene | Protein | Location |
|---|---|---|---|---|---|
| 1 | 11753127_a_at | Aryl Hydrocarbon | ARNTL | Aryl hydrocarbon receptor | Intracellular Membrane-Bounded |
| | | Receptor Nuclear | | nuclear translocator-like | Organelle |
| | | Translocator Like | | protein 1 | |
| 2 | 11758490_s_at | Spartin | SPART | Spartin | Membrane, Cytoplasm |
| 3 | 11739116_s_at | EFR3 Homolog A | EFR3A | Protein EFR3 homolog A | Membrane Bound (Lipid-Anchor), |
| | | | | | Cytosol |
| 4 | 11762833_x_at | mRNA/cDNA for | LOC642846 | None Known | Unknown |
| | | LOC642846 gene | | | |
| | | (DEAD/H (Asp-Glu- | | | |
| | | Ala-Asp/His) box | | | |
| | | polypeptide 11-like) | | | |
| 5 | 11717380_x_at | FGFR1OP N- | FOPNL | Centrosomal protein 20 | Cytoskeleton, Nucleus |
| | | Terminal Like | | | |
| 6 | 11730494_x_at | Potassium Two Pore | KCNK2 | Potassium channel subfamily | Membrane (Multi-pass) |
| | | Domain Channel | | K member 2 | |
| | | Subfamily K | | | |
| | | Member 2 | | | |
| 7 | 11746418_a_at | Vitrin | VIT | Vitrin | Secreted |
| 8 | 11722500_a_at | LysM Domain | LYSMD3 | LysM and putative | Membrane (Single-pass)/Golgi |
| | | Containing 3 | | peptidoglycan-binding | Apparatus |
| | | | | domain-containing protein 3 | |
| 9 | 11737591_a_at | MybLike, SWIRM | MYSM1 | Histone H2A deubiquitinase | Nucleus |
| | | And MPN Domains 1 | | MYSM1 | |
| 10 | 11724280_a_at | Inhibitor of Growth | ING3 | Inhibitor of growth protein 3 | Nucleus |
| | | Family Member 3 | | | |
| 11 | 11723439_at | Sushi Repeat | SRPX2 | Sushi repeat-containing | Secreted |
| | | Containing Protein | | protein SRPX2 | |
| | | X-Linked 2 | | | |
| 12 | 11756516_a_at | Phosphatidylserine | PISD | Phosphatidylserine | Membrane (Single-pass)/Mitochondrion |
| | | Decarboxylase | | decarboxylase proenzyme, | |
| | | | | mitochondrial | |
| 13 | 11738195_a_at | Fibroblast Growth | FGF1 | Fibroblast growth factor 1 | Secreted |
| | | Factor 1 | | | |
| 14 | 11740395_a_at | Fibroblast Growth | FGFR2 | Fibroblast growth factor | Membrane (Single-pass Type I)/Golgi |
| | | Factor Receptor 2 | | receptor 2 | Apparatus |

TABLE 2B
mRNA and Protein Biomarkers: NAFL v. NASH

| No. | Marker Probe ID | Description | Gene | Protein | Location |
|---|---|---|---|---|---|
| 1 | 11733218_at | Transcription Factor | TFAp4 | Transcription factor AP-4 | Nucleus |
| | | AP-4 | | | |
| 2 | 11723753_a_at | Zinc Finger AN1-Type | ZFAND2A | AN1-type zinc finger protein | Nucleus & Cytoplasm |
| | | Containing 2A | | 2A | |
| 3 | 11722163_x_at | Polo Like Kinase 2 | PLK2 | Serine/threonine-protein | Centriole |
| | | | | kinase PLK2 | |
| 4 | 11758103_s_at | Transmembrane P24 | TMED3 | Transmembrane emp24 | Golgi Apparatus & Endoplasmic |
| | | Trafficking Protein 3 | | domain-containing protein 3 | Reticulum |
| 5 | 11724489_s_at | Aldo-Keto Reductase | AKR1B10 | Aldo-keto reductase family 1 | Secreted |
| | | Family 1 Member B10 | | member B10 | |
| 6 | 11732809_a_at | Prokineticin 2 | PROK2 | Prokineticin-2 | Secreted |
| 7 | 11723106_a_at | Neutrophil Cytosolic | NCF4 | Neutrophil cytosol factor 4 | Membrane Bound (Peripheral), |
| | | Factor 4 | | | Cytosol, Endosome |
| 8 | 11747524_a_at | Galactosylceramidase | GALC | Galactocerebrosidase | Lysosome |
| 9 | 11732727_a_at | SLIT And NTRK Like | SLITRK4 | SLIT and NTRK-like protein 4 | Membrane (Single-pass Type I) |
| | | Family Member 4 | | | |

TABLE 3B
mRNA and Protein Biomarkers: NASH v. Healthy Liver

| No. | Marker Probe ID | Description | Gene | Protein | Location |
|---|---|---|---|---|---|
| 1 | 16665621 | mRNA: CACHD1 | CACHD1 | VWFA and cache domain- | Membrane (Single-pass Type I) |
| | | | | containing protein 1 | |
| 2 | 16669169 | mRNA: CD2 molecule, | CD2 | T-cell surface antigen CD2 | Membrane (Single-pass Type I) |
| | | transcript variant 2 | | | |
| 3 | 16796773 | chr14: 101798302- | None Known | None Known | Unknown |
| | | 101801206 | | | |
| 4 | 16829985 | mRNA: ENO3 gene | ENO3 | Beta-enolase | Secreted |
| 5 | 16886466 | chr2: 151026010- | None Known | None Known | Unknown |
| | | 151157594 | | | |
| 6 | 16890891 | mRNA: VIL1 gene | VIL1 | Villin-1 | Secreted |
| 7 | 16754913 | chr12: 88210263- | None Known | None Known | Unknown |
| | | 88211608 | | | |
| 8 | 17048083 | chr7: 89796904- | None Known | None Known | Unknown |
| | | 89870091 | | | |
| 9 | 16766132 | apolipoprotein F | APOF | Apolipoprotein F | Secreted |
| | | (APOF), mRNA | | | |
| 10 | 16673227 | chr1: 164608802- | None Known | None Known | Unknown |
| | | 164608908 | | | |
| 11 | 16818272 | chr16: 31366509- | ITGAX | Integrin alpha-X | Membrane (Single-pass Type I) |
| | | 31394318 | | | |
| 12 | 16977052 | C-X-C motif chemokine | CXCL10 | C-X-C motif chemokine 10 | Secreted |
| | | ligand 10 (CXCL10), | | | |
| | | mRNA | | | |
| 13 | 16706008 | nudixhydrolase 13 | NUDT13 | Nucleoside diphosphate- | Mitochondrion |
| | | (NUDT13), transcript | | linked moiety X motif 13 | |
| | | variant 1, mRNA | | | |
| 14 | 16834015 | Rap guanine nucleotide | RAPGEFL1 | Rap guanine nucleotide | Membrane |
| | | exchange factor like 1 | | exchange factor-like 1 | |
| | | (RAPGEFL1), transcript | | | |
| | | variant 3, mRNA | | | |
| 15 | 17106795 | SH2 domain containing | SH2D1A | SH2 domain-containing | Cytoplasm |
| | | 1A (SH2D1A), transcript | | protein 1A | |
| | | variant 1, mRNA | | | |
| 16 | 16714880 | chr10: 69556048- | None Known | None Known | Unknown |
| | | 69597937 | | | |
| 17 | 16851866 | chr18: 32073254- | None Known | None Known | Unknown |
| | | 32471808 | | | |
| 18 | 16830607 | sex hormone binding | SHBG | Sex hormone-binding | Secreted |
| | | globulin (SHBG), | | globulin | |
| | | transcript variant 1, | | | |
| | | mRNA | | | |
| 19 | 16659443 | Prame Family Member | PRAMEF17 | PRAME family member 17 | Cytoplasm |
| | | 17 (PRAMEF17), mRNA | | | |
| 20 | 16660810 | RCAN Family Member | RCAN3 | Calcipressin-3 | Cytoplasm |
| | | 3, (RCAN3), mRNA | | | |
| 21 | 16669963 | G Protein-Coupled | GPR89A | Golgi pH regulator A | Membrane (Multi-pass), Golgi |
| | | Receptor 89A (GPR89A), | | | Apparatus |
| | | mRNA | | | |
| 22 | 16745651 | Olfactory Receptor | OR8B8 | Olfactory receptor 8B8 | Membrane (Multi-pass) |
| | | Family 8 Subfamily B | | | |
| | | Member 8 (OR8B8), | | | |
| | | mRNA | | | |
| 23 | 16811975 | Tetraspanin3 (TSPAN3), | TSPAN3 | Tetraspanin-3 | Membrane (Multi-pass) |
| | | mRNA | | | |
| 24 | 16854594 | GRB2 Associated | GAREM1 | GRB2-associated and | Membrane (Plasma) |
| | | Regulator Of MAPK1 | | regulator of MAPK protein 1 | |
| | | Subtype 1 (GAREM1), | | | |
| | | mRNA | | | |
| 25 | 16903953 | Activin A Receptor Type | ACVR1C | Activin receptor type-1C | Membrane (Single-pass Type I) |
| | | 1C (ACVR1C), mRNA | | | |
| 26 | 16934434 | Apolipoprotein L3 | APOL3 | Apolipoprotein L3 | Secreted |
| | | (APOL3), mRNA | | | |
| 27 | 17031373 | Ubiquitin D (FAT10), | FAT10 (aka | Ubiquitin D | Nucleus, Cytoplasm |
| | | mRNA | UBD) | | |
| 28 | 16909401 | Solute Carrier Family 16 | SLC16A14 | Monocarboxylate | Membrane (Multi-pass) |
| | | Member 14 (SLC16A14), | | transporter 14 | |
| | | mRNA | | | |
| 29 | 17108996 | Family with Sequence | FAM9B | Protein FAM9B | Nucleus |
| | | Similarity 9 Member B | | | |
| | | (FAM9B), mRNA | | | |
| 30 | 17020964 | Interphotoreceptor | IMPG1 | Interphotoreceptor matrix | Secreted |
| | | Matrix Proteoglycan | | proteoglycan 1 | |
| | | 1(IMPG1), mRNA | | | |
| 31 | 16977045 | C-X-C Motif Chemokine | CXCL9 | C-X-C motif chemokine 9 | Secreted |
| | | Ligand 9 (CXCL9), mRNA | | | |
| 32 | 16677407 | SET And MYND Domain | SMYD2 | N-lysine methyltransferase | Nucleus, Cytosol |
| | | Containing 2 (SMYD2), | | SMYD2 | |
| | | mRNA | | | |
| 33 | 16695535 | Rho GTPase Activating | ARHGAP30 | Rho GTPase-activating | Intracellular Membrane-Bounded |
| | | Protein 30 (ARHGAP30), | | protein 30 | Organelle |
| | | mRNA | | | |
| 34 | 16672654 | SLAM Family Member 7 | SLAMF7 | SLAM family member 7 | Membrane (Single-pass Type I) |
| | | (SLAMF7), mRNA | | | |
| 35 | 16859395 | Myosin IXB (MYO9B), | MYO9B | Unconventional myosin-IXb | Cytoskeleton |
| | | mRNA | | | |
| 36 | 16661149 | CD52 molecule (CD52), | CD52 | CAMPATH-1 antigen | Membrane Bound (GPI-Anchor) |
| | | mRNA | | | |
| 37 | 17074914 | Macrophage Scavenger | MSR1 | Macrophage scavenger | Membrane (Single-pass Type II) |
| | | Receptor 1 (MSR1), a | | receptor types I and II | |
| | | transcript of gene | | | |
| | | ENSG00000038945.15 | | | |

1.-101. (canceled)

102. A method of diagnosing or determining a prognosis of a liver disease in a subject, the method comprising:
(a) measuring expression levels of one or more biomarkers in a sample from the subject;
(b) receiving the expression levels of the one or more biomarkers in the subject sample with a memory component comprising a computer-executable program;
(c) comparing the expression levels of the one or more biomarkers in the subject sample to a level in a control sample for the same one or more biomarkers;
(d) receiving a result comparing the expression levels of the one or more biomarkers in the subject sample measured in (a) and in the control sample measured in (c);
(e) diagnosing or determining the prognosis of the liver disease based on the compared expression of the one or more biomarkers in the subject sample as compared to the control sample determined by the memory component; and
(f) providing a treatment plan for the subject having liver disease based on the diagnosis or prognosis determined in (e).

103. The memory component of claim 102, wherein the liver disease is at least one or more of nonalcoholic fatty liver (NAFL), nonalcoholic steatohepatitis (NASH), hepatitis B, and hepatitis C.

104. The method of claim 103, wherein the NAFL comprises NAFLD stage 1, NAFLD stage 2, NAFLD stage 3, or NAFLD stage 4.

105. The method of claim 102, wherein the one or more biomarkers comprises one or more miRNAs selected from the group consisting of hsa-let-7d, hsa-miR-149, hsa-miR-513a-3p, hsa-miR-192, hsa-miR-23b, hsa-miR-301a, hsa-miR-933, hsa-miR-148a, hsa-miR-17, hsa-miR-423-3p, hsa-miR-563, hsa-miR-596, hsa-miR-150, hsa-mi R-1260, hsa-miR-1539, hsa-miR-15b, hsa-mi R-887, hsa-mi R-602, hsa-mi R-610, hsa-miR-1237, hsa-miR-20b, hsa-miR-425, hsa-miR-718, hsa-miR-595, hsa-miR-101, hsa-miR-483-3p, hsa-miR-103, hsa-miR-186, hsa-miR-33b, hsa-miR-1225-3p, hsa-miR-331-3p, and has-miR-215.

106. The method of claim 102, wherein the one or more biomarkers comprise:
(a) mRNAs encoded by the genes ARNTL, SPART, EFR3A, LOC642846, FOPNL, KCNK2, VIT, LYSMD3, MYSM1, ING3, SRPX2, PISD, FGF1, and/or FGFR2;
(b) mRNAs encoded by the genesTFAp4, ZFAND2A, PLK2, TMED3, AKR1B10, PROK2, NCF4, GALC, and/or SLITRK4; or
(c) mRNAs encoded by the genes CACHD1, CD2 molecule, transcript variant 2, chr14:101798302-101801206, ENO3 gene, chr2:151026010-151157594, VIL1 gene, chr12:88210263-88211608, chr7:89796904-89870091, apolipoprotein F (APOF), chr1:164608802-164608908, chr16:31366509-31394318, C-X-C motif chemokine ligand (CXCL10), nudixhydrolase 13 (NUDT13), transcript variant 1, Rap guanine nucleotide exchange factor like 1 (RAPGEFL1), transcript variant 3, SH2 domain containing 1A (SH2D1A), transcript variant 1, chr10:69556048-69597937, chr18:32073254-32471808, sex hormone binding globulin (SHBG), transcript variant 1, Prame Family Member 17 (PRAMEF17), RCAN Family Member 3, (RCAN3), G Protein-Coupled Receptor 89A (GPR89A), Olfactory Receptor Family 8 Subfamily B Member 8 (OR8B8), Tetraspanin3 (TSPAN3), GRB2 Associated Regulator of MAPK1 Subtype 1 (GAREM1), Activin A Receptor Type 1C (ACVR1C), Apolipoprotein L3 (APOL3), Ubiquitin D (FAT10), Solute Carrier Family 16 Member 14 (SLC16A14), Family with Sequence Similarity 9 Member B (FAM9B), Interphotoreceptor Matrix Proteoglycan 1(IMPG1), C-X-C Motif Chemokine Ligand 9 (CXCL9), SET And MYND Domain Containing 2 (SMYD2), Rho GTPase Activating Protein 30 (ARHGAP30), SLAM Family Member 7 (SLAMF7), Myosin IXB (MYO9B), CD52 molecule (CD52), and/or Macrophage Scavenger Receptor 1 (MSR1).

107. The method of claim 102, wherein the computer-executable program comprises a processing module and an analysis module.

108. The method of claim 107, wherein the processing module comprises an algorithm for measuring the expression levels of the one or more biomarkers in the subject sample.

109. The method of claim 107, wherein the analysis module comprises an algorithm for comparing the expression levels of the one or more biomarkers in the subject sample relative to the expression of the same one or more biomarkers in the control sample.

110. The method of claim 102, wherein the one or more biomarkers are comprised of (a) at least 2, 3, 4, 5, 6, 7, 8, 9, 10, 11, 12, 13, and/or 14 biomarkers; (b) at least 2, 3, 4, 5, 6, 7, 8, and/or 8 biomarkers; or (c) at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, and/or 37 biomarkers.

111. The method of claim 102, wherein the one or more biomarkers are comprised of (a) no more than 2, 3, 4, 5, 6, 7, 8, 9, 10, 11, 12, 13, and/or 14 biomarkers; (b) no more than 2, 3, 4, 5, 6, 7, 8, and/or 8 biomarkers; or (c) no more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, and/or 37 biomarkers.

112. A memory component with instructions stored thereon that, when executed by a processor of a computing device, causes the computing device to perform steps for determining expression levels of one or more biomarkers in a sample from a subject, comprising the steps of:
(i) providing, to the sample, a processing module for determining an expression level of each of the one or more biomarkers;
(ii) normalizing the expression levels of the one or more biomarkers in (i) relative to an expression level of the same one or more biomarkers in a control sample;
(iii) calculating one or more threshold values based on the normalized expression levels of the one or more biomarkers in (ii) relative to the expression level of the same one or more biomarkers in the control sample;
(iv) creating a score from the normalized expression levels of the one or more biomarkers in (ii), wherein the score indicates whether the expression levels of the one or more biomarkers are altered relative to the control, and
(v) providing, to the normalized expression levels, an analysis module for determining a liver disease status and/or a diagnosis of liver disease in the subject.

113. The memory component of claim 112, wherein the subject has a liver disease or is suspected to have the liver disease.

114. The memory component of claim 113, wherein the liver disease is at least one or more of nonalcoholic fatty liver (NAFL), nonalcoholic steatohepatitis (NASH), hepatitis B, and hepatitis C.

115. The memory component of claim 114, wherein the NAFL comprises NAFLD stage 1, NAFLD stage 2, NAFLD stage 3, or NAFLD stage 4.

116. The memory component of claim 112, wherein the one or more biomarkers comprises one or more miRNAs are selected from the group consisting of hsa-let-7d, hsa-miR-149, hsa-miR-513a-3p, hsa-miR-192, hsa-miR-23b, hsa-miR-301a, hsa-miR-933, hsa-miR-148a, hsa-miR-17, hsa-miR-423-3p, hsa-miR-563, hsa-miR-596, hsa-mi R-150, hsa-mi R-1260, hsa-mi R-1539, hsa-mi R-15b, hsa-mi R-887, hsa-mi R-602, hsa-miR-610, hsa-miR-1237, hsa-miR-20b, hsa-miR-425, hsa-miR-718, hsa-miR-595, hsa-miR-101, hsa-miR-483-3p, hsa-miR-103, hsa-miR-186, hsa-miR-33b, hsa-miR-1225-3p, hsa-miR-331-3p, and has-miR-215.

117. The memory component of claim 116, wherein the one or more miRNAs are comprised of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, and/or 32 biomarkers.

118. The memory component of claim 116, wherein the one or more miRNAs are comprised of no more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, and/or 32 biomarkers.

119. The memory component of claim 112, wherein the one or more biomarkers comprise:
(a) mRNAs encoded by the genes ARNTL, SPART, EFR3A, LOC642846, FOPNL, KCNK2, VIT, LYSMD3, MYSM1, ING3, SRPX2, PISD, FGF1, and/or FGFR2;
(b) mRNAs encoded by the genesTFAp4, ZFAND2A, PLK2, TMED3, AKR1B10, PROK2, NCF4, GALC, and/or SLITRK4; or
(c) mRNAs encoded by the genes CACHD1, CD2 molecule, transcript variant 2, chr14:101798302-101801206, ENO3 gene, chr2:151026010-151157594, VIL1 gene, chr12:88210263-88211608, chr7:89796904-89870091, apolipoprotein F (APOF), chr1:164608802-164608908, chr16:31366509-31394318, C-X-C motif chemokine ligand 10 (CXCL10), nudixhydrolase 13 (NUDT13), transcript variant 1, Rap guanine nucleotide exchange factor like 1 (RAPGEFL1), transcript variant 3, SH2 domain containing 1A (SH2D1A), transcript variant 1, chr10:69556048-69597937, chr18:32073254-32471808, sex hormone binding globulin (SHBG), transcript variant 1, Prame Family Member 17 (PRAMEF17), RCAN Family Member 3, (RCAN3), G Protein-Coupled Receptor 89A (GPR89A), Olfactory Receptor Family 8 Subfamily B Member 8 (OR8B8), Tetraspanin3 (TSPAN3), GRB2 Associated Regulator of MAPK1 Subtype 1 (GAREM1), Activin A Receptor Type 1C (ACVR1C), Apolipoprotein L3 (APOL3), Ubiquitin D (FAT10), Solute Carrier Family 16 Member 14 (SLC16A14), Family with Sequence Similarity 9 Member B (FAM9B), Interphotoreceptor Matrix Proteoglycan 1(IMPG1), C-X-C Motif Chemokine Ligand 9 (CXCL9), SET And MYND Domain Containing 2 (SMYD2), Rho GTPase Activating Protein 30 (ARHGAP30), SLAM Family Member 7 (SLAMF7), Myosin IXB (MYO9B), CD52 molecule (CD52), and/or Macrophage Scavenger Receptor 1 (MSR1).

120. The memory component of claim 119, wherein the one or more mRNAs are comprised of (a) at least 2, 3, 4, 5, 6, 7, 8, 9, 10, 11, 12, 13, and/or 14 mRNAs; (b) at least 2, 3, 4, 5, 6, 7, 8, and/or 8 mRNAs; or (c) at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 36, and/or 37 mRNAs.

121. The memory component of claim 119, wherein the one or more mRNAs are comprised of (a) no more than 2, 3, 4, 5, 6, 7, 8, 9, 10, 11, 12, 13, and/or 14 mRNAs; (b) no more than 2, 3, 4, 5, 6, 7, 8, and/or 8 mRNAs; or (c) no more than 2, 3, 4, 6, 7, 8, 9, 10, 11, 12, 13, 14, 15, 16, 17, 18, 19, 20, 21, 22, 23, 24, 25, 26, 27, 28, 29, 31, 32, 33, 34, 35, 36, and/or 37 mRNAs.
